(19) United States
(12) Patent Application Publication
(10) Pub. No.: US 2010/0029675 A1
(43) Pub. Date: Feb. 4, 2010

(54) Pyrimidine-2, 4-diamine JAK2 Kinase inhibiting anti-inflammation use

(76) Inventor: Soo-In Hwang, San Carlos, CA (US)

Correspondence Address: SuperGen, Inc., 4140 Dublin Blvd., Suite 200, Dublin, CA, 94568, US

(21) Appl. No.: 12/460,820

(22) Filed: Jul. 23, 2009

Related U.S. Application Data
(60) Provisional application No. 61/137,040, filed on Jul. 25, 2008.

(51) Int. Cl. A61K 31/496; A61P 9/10; A61P 11/06; A61P 11/00; A61P 3/00; A61P 31/00; A61P 29/00; A61P 19/02; A61P 31/10; A61P 31/12; A61P 31/04; A61P 17/00; A61P 27/06

(52) U.S. Cl. 514/252.18; 514/252.14

(57) ABSTRACT

A method of treatment or prevention of Castleman's disease, atherosclerosis, coronary artery disease, peripheral edema, peripheral vascular disease, glaucoma, and wet or dry age-related macular degeneration (AMD), asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration comprises the step of administering a therapeutically effective amount, or a prophylactically effective amount, of a compound having the following structure (I):

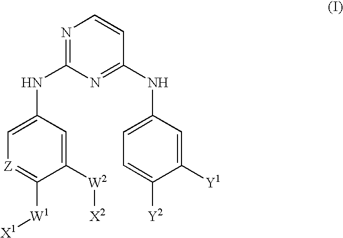

This application claims the benefit of U.S. Patent Application No. 61/137,040, filed July 25, 2008.

### BACKGROUND OF THE INVENTION

The present invention is directed to compounds that inhibit protein kinase activity, and to compositions and methods related thereto their use in anti-inflammation, including against rheumatoid arthritis.

The Janus kinases (JAKs) are a family of four in mammals (JAK1, JAK2, JAK3 and TYK2) integral in signaling from extracellular cytokines, including the interleukins, interferons, as well as numerous hormones (Aringer, M., et al., Life Sci, 1999. 64(24): p. 2173-86; Briscoe, J., et al., Philos Trans R Soc Lond B Biol Sci, 1996. 351(1336): p. 167-71; Ihle, J. N., Semin Immunol, 1995. 7(4): p. 247-54; Ihle, J. N., Philos Trans R Soc Lond B Biol Sci, 1996. 351(1336): p. 159-66; Firmbach-Kraft, I., et al., Oncogene, 1990. 5(9): p. 1329-36; Harpur, A. G., et al., Oncogene, 1992. 7(7): p. 1347-53; Rane, S. G. and E. P. Reddy, Oncogene, 1994. 9(8): p. 2415-23; Wilks, A. F., Methods Enzymol, 1991. 200: p. 533-46). These non-receptor tyrosine kinases associate with various cytokine receptors and act to transduce the signal from extracellular ligand-receptor binding into the cytoplasm, by phosphorylating STAT (signal transducer and activator of transcription) molecules, which then enter the nucleus and direct transcription of various target genes involved in growth and proliferation (Briscoe, J., et al.; Ihle, J. N. (1995); Ihle, J. N. (1996); Rawlings, J. S., K. M. Rosler and D. A. Harrison, J Cell Sci, 2004. 117(Pt 8): p. 1281-3.). The importance of these kinases in cellular survival is made evident by the fact that the loss of JAKs is often accompanied by immunodeficiency and non-viability in animal models (Aringer, M., et al.). The JAK family of enzymes is characterized by a number of JAK homology (JH) domains, including a carboxy-terminal protein tyrosine kinase domain (JH1) and an adjacent kinase-like domain (JH2), which is thought to regulate the activity of the JH1 domain (Harpur, A. G., et al.). The four JAK isoforms transduce different signals by being associated specifically with certain cytokine receptors, and activating a subset of downstream genes. For example, JAK2 associates with cytokine receptors specific for interleukin-3 (Silvennoinen, O., et al., Proc Natl Acad Sci USA, 1993. 90(18): p. 8429-33), erythropoietin (Witthuhn, B. A., et al., Cell, 1993. 74(2): p. 227-36), granulocyte colony stimulating factor (Nicholson, S. E., et al., Proc Natl Acad Sci USA, 1994. 91(8): p. 2985-8), and growth hormone (Argetsinger, L. S., et al., Cell, 1993. 74(2): p. 237-44).

The JAK family of enzymes has become an interesting set of targets for various hematological and immunological disorders; JAK2 specifically is currently under study as a viable target for neoplastic disease, especially leukemias and lymphomas (Benekli, M., et al., Blood, 2003. 101(8): p. 2940-54; Peeters, P., et al., Blood, 1997. 90(7): p. 2535-40; Reiter, A., et al., Cancer Res, 2005. 65(7): p. 2662-7; Takemoto, S., et al., Proc Natl Acad Sci USA, 1997. 94(25): p. 13897-902) as well as solid tumors (Walz, C., et al., J Biol Chem, 2006. 281(26): p. 18177-83), and other myeloproliferative disorders such as polycythemia vera (Baxter, E. J., et al., Lancet, 2005. 365(9464): p. 1054-61; James, C., et al., Nature, 2005. 434(7037): p. 1144-8; Levine, R. L., et al., Cancer Cell, 2005. 7(4): p. 387-97; Shannon, K. and R. A. Van Etten, Cancer Cell, 2005. 7(4): p. 291-3), due to its activation of downstream effector genes involved in proliferation. Because of its association with, and deregulation in, neoplastic and myeloproliferative disorders, small molecule JAK2 inhibitors for the treatment of human malignancies are of significant interest.

Inflammation is a critical process involved in the genesis and maintenance of coronary artery disease, metabolic disease, diabetes, obesity, and other cardiovascular diseases. JAK/STAT signaling mediates many of the critical components involved in these disease processes. Peripheral edema implicates molecular pathways involving JAK/STAT signaling such as VEGF and HGF similarly to the pathways' involvement in oncology, cardiovascular, and metabolic syndromes. In vascular pathobiology, JAK/STAT mediates inflammatory signals involved in cell proliferation, angiogenesis or neovascularization, and development of the atherosclerotic lesions as well as peripheral edema. JAK/STAT signaling regulates cytokine mediated generation of inflammatory helper T cells.

JAK/STAT is involved in mediating the signaling of many inflammatory cytokines involved in inflammation and proliferation including: IL2, IL3, IL4, IL6, IL7, IL9, IL10, IL11, IL12, IL13, IL15, IL17, IL23, PDGF, IFNa, IFNb, and IFNg. For example, both HGF and VEGF induce IL6, which signals through the JAK/STAT complex to carry out their inflammation and angiogenesis functions. Hence, an inhibitor of JAK kinases such as compounds set forth in this invention, which inhibits JAK 1, JAK 2, and Tyk, will be useful in many inflammatory disease processes involved in the pathogenesis of atherosclerosis, coronary artery disease, peripheral edema, peripheral vascular disease, and other cardiovascular pathologies involving the JAK/STAT mechanism of action, as well as glaucoma, and wet or dry age-related macular degeneration (AMD) in which HGF and VEGF are also implicated.

Thus, JAK2 inhibition as described above, affects the cytokines and would affect afflictions such as asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration, which are associated with cytokine activity. JAK2 inhibition also would be of benefit in the treatment of nonmalignant tumors such as, for example, Castleman's disease.

Accordingly, there remains a need for efficacious small molecule compounds that mediate cytokine activity through JAK2 inhibition in order to treat or prevent such diseases associated with cytokine activity.

### SUMMARY OF THE INVENTION

A method of treatment or prevention of Castleman's disease, atherosclerosis, coronary artery disease, peripheral edema, peripheral vascular disease, glaucoma, and wet or dry age-related macular degeneration (AMD), asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration comprises the step of administering a therapeutically effective amount, or a prophylactically effective amount, of a compound having the following structure (I) or (II):

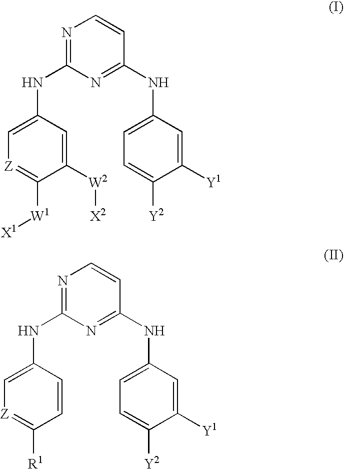

including stereoisomers, prodrugs and pharmaceutically acceptable salts thereof, wherein R¹, W¹, W², X¹, X², Y¹, Y² and Z are as defined herein.

These compounds have utility over a broad range of therapeutic applications, and may be used to treat diseases, such as asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration, that are mediated and/or associated (at least in part) with JAK2 protein kinase. Accordingly, in one aspect of the invention, the compounds described herein are formulated as pharmaceutically acceptable compositions for administration to a subject in need thereof.

In another aspect, the invention provides methods for treating or preventing a JAK2 protein kinase-mediated disease, such as asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration, which method comprises administering to a patient in need of such a treatment a therapeutically effective amount of a compound described herein or a pharmaceutically acceptable composition comprising said compound.

Another aspect relates to inhibiting JAK2 protein kinase activity in a biological sample, which method comprises contacting the biological sample with a compound described herein, or a pharmaceutically acceptable composition comprising said compound.

Another aspect relates to a method of inhibiting JAK2 protein kinase activity in a patient, which method comprises administering to the patient a compound described herein or a pharmaceutically acceptable composition comprising said compound.

These and other aspects of the invention will be apparent upon reference to the following detailed description. To that end, certain patent and other documents are cited herein to more specifically set forth various aspects of this invention. Each of these documents is hereby incorporated by reference in its entirety.

### DETAILED DESCRIPTION OF THE INVENTION

According to a general aspect of the present invention, there are provided compounds useful as JAK2 protein kinase inhibitors, as well as compositions and methods relating thereto. Compounds of the invention have the structure set forth in formula (I) or (II):

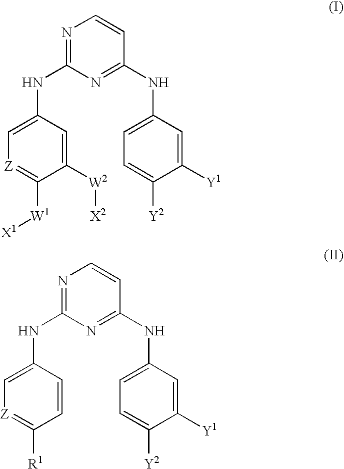

including stereoisomers and pharmaceutically acceptable salts thereof, wherein:

### Z is CH or N;

W¹ and W² are independently a direct bond, —C(═O)— or —O(CH₂)ₙ—;
R¹ is —H, —CF₃, —OCF₃, —OCHF₂, —OCH₃, —CH₃, —OH, —NO₂, —NH₂ or halogen;
X¹ and X² are independently —H, —CF₃, —OCF₃, —OCHF₂, —OCH₃, —CH₃, —OH, —NO₂, —NH₂, halogen or

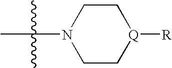

wherein Q is O or N and R is not present or R is —C₁₋₆alkyl, provided that one of X¹ or X² is

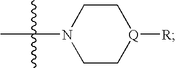

Y¹ and Y² are independently —H, —CN, halogen or a C₁₋₄alkyl group substituted with —CN, provided that Y¹ and Y² are not both —H; and
n is 1, 2 or 3.

Unless otherwise stated the following terms used in the specification and claims have the meanings discussed below:

“Halogen” means fluoro, chloro, bromo, or iodo, and typically fluoro or chloro.

“C₁₋₆alkyl” refers to a saturated straight or branched, saturated or unsaturated, cyclic or noncyclic hydrocarbon radical of one to six carbon atoms, while “C₁₋₄alkyl” has the same meaning but contains one to four carbon atoms. Representative examples of saturated straight chain or branched C₁₋₄alkyls and C₁₋₆alkyls include methyl, ethyl, n-propyl, isopropyl, n-butyl, isobutyl, sec-butyl, and tert-butyl, and in the case of C₁₋₆alkyls further include n-pentyl, n-hexyl, and corresponding branched chains. Representative saturated cyclic alkyls include cyclopropyl, cyclobutyl, cyclopentyl, —CH₂cyclopentyl, cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

In more specific aspects of structures (I) and (II) above, Z is CH and the compounds are represented by the following structures (III) or (IV):

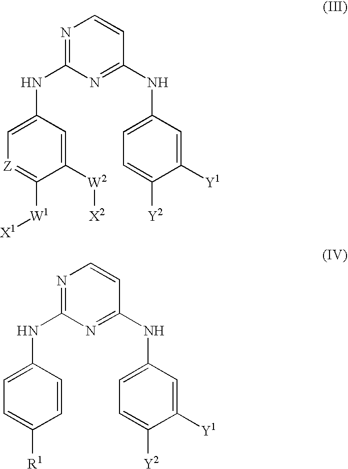

including stereoisomers and pharmaceutically acceptable salts thereof.

In other aspects of structures (I) and (II) above, Z is N and the compounds are represented by the following structures (V) or (VI):

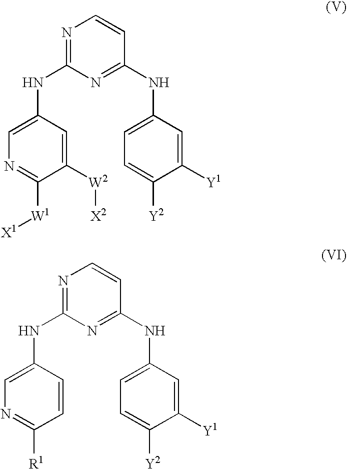

including stereoisomers and pharmaceutically acceptable salts thereof.

In more specific embodiments of structures (III) and (V), X¹ is piperazinyl, R is methyl and W¹ and W² are both direct bonds and the compounds are represented by structures (VII) and (VIII), respectively:

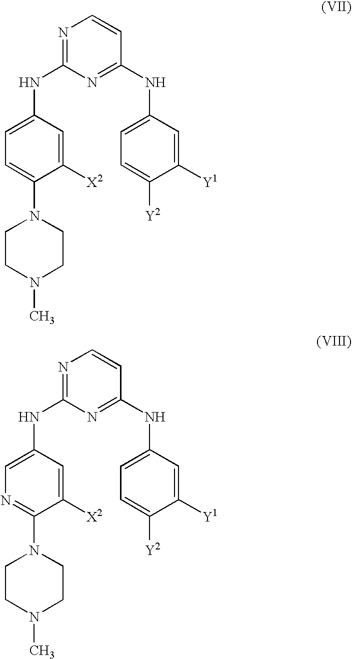

In more specific embodiments of structures (VII) and (VIII), X² is —F, and the compounds are represented by structures (IX) and (X), respectively:

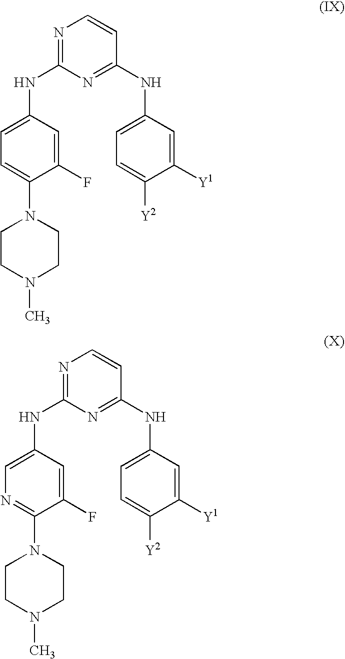

In other further specific embodiments of structures (VII) and (VIII), X² is —H, and the compounds are represented by structures (XI) and (XII), respectively:

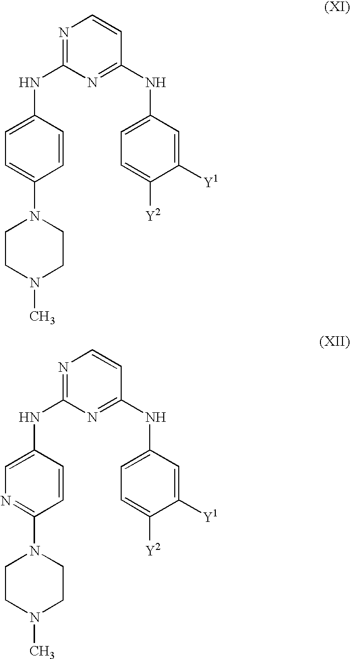

In other specific embodiments of structure (III), X² is piperazinyl, R is methyl and W² is —O(CH₂)₃— and W¹ is a direct bond and the compounds are represented by structure (XIII):

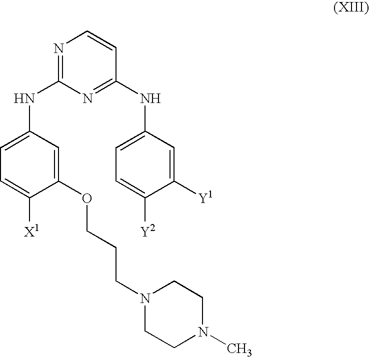

In further specific embodiments of structure (XIII), X¹ is —OCH₃.

In other specific embodiments of structure (III), X¹ is piperazinyl, W¹ is —C(═O)— and W² is a direct bond and the compounds are represented by structure (XIV):

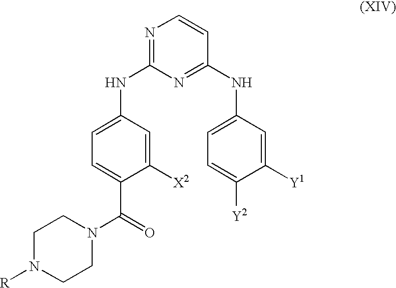

In further specific embodiments of structure (XIV), X² is —H and R is methyl or R is cyclohexyl.

In specific embodiments of structures (I), (II), (III), (IV), (V), (VI), (VII), (VIII), (IX), (X), (XI), (XII), (XIII), and (XIV), Y¹ and Y² are halogen, and more specifically Y¹ is chloro and Y² is fluoro.

In specific embodiments of structures (I), (III), (V), (VII), (VIII), (IX), (X), (XI), (XII), Y¹ and Y² are —CN and halogen, and more specifically Y¹ is CN and Y² is fluorine.

In specific embodiments of structures (I), (III), (IV), (V), (VI), (VII), (VIII), (IX), (X), (XI), (XII), (XIII), and (XIV), Y¹ and Y² are —H and a C₁₋₄alkyl group substituted with —CN, and more specifically Y¹ is —CH₂CN and Y² is —H.

In specific embodiments of structures (II), (IV) and (VI), R¹ is —F or R¹ is —CF₃.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog (Cahn, R., Ingold, C., and Prelog, V. Angew. Chem. 78:413-47, 1966; Angew. Chem. Internat. Ed. Eng. 5:385-415, 511, 1966), or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Ch. 4 of ADVANCED ORGANIC CHEMISTRY, 4^{th} edition, March, J., John Wiley and Sons, New York City, 1992).

The compounds of the present invention may exhibit the phenomena of tautomerism and structural isomerism. This invention encompasses any tautomeric or structural isomeric form and mixtures thereof which possess the ability to modulate JAK2-2 kinase activity and is not limited to, any one tautomeric or structural isomeric form.

It is contemplated that a compound of the present invention would be metabolized by enzymes in the body of the organism such as human being to generate a metabolite that can modulate the activity of the protein kinases. Such metabolites are within the scope of the present invention.

A compound of the present invention or a pharmaceutically acceptable salt thereof, can be administered as such to a patient, including a human, or can be administered in pharmaceutical compositions in which the foregoing materials are mixed with suitable carriers or excipient(s). Techniques for formulation and administration of drugs may be found, for example, in REMINGTON'S PHARMACOLOGICAL SCIENCES, Mack Publishing Co., Easton, Pa., latest edition.

A “pharmaceutical composition” refers to a mixture of one or more of the compounds described herein, or pharmaceutically acceptable salts thereof, with other chemical components, such as pharmaceutically acceptable excipients. The purpose of a pharmaceutical composition is to facilitate administration of a compound to an organism.

“Pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Pharmaceutically acceptable salt” refers to those salts which retain the biological effectiveness and properties of the parent compound. Such salts may include: (1) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

The term “JAK2 protein kinase-mediated condition” or “disease”, as used herein, means any disease or other deleterious condition in which a JAK2 protein kinase is known to play a role. The term “JAK2 protein kinase-mediated condition” or “disease” also means those diseases or conditions that are alleviated by treatment with a JAK2 protein kinase inhibitor, including cancer as discussed in greater detail below.

As used herein, “administer” or “administration” refers to the delivery of an inventive compound or of a pharmaceutically acceptable salt thereof or of a pharmaceutical composition containing an inventive compound or a pharmaceutically acceptable salt thereof of this invention to an organism for the purpose of prevention or treatment of a protein kinase-related disorder.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. In certain embodiments, the routes of administration are oral and intravenous.

Alternatively, one may administer the compound in a local rather than systemic manner, for example, via injection of the compound directly into a solid tumor, often in a depot or sustained release formulation.

Furthermore, one may administer the compound in a targeted delivery system, for example, in a liposome coated with tumor-specific antibody. In this way, the liposomes may be targeted to and taken up selectively by the tumor.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in any conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also. Pharmaceutical compositions which may also be used include hard gelatin capsules. The capsules or pills may be packaged into brown glass or plastic bottles to protect the active compound from light. The containers containing the active compound capsule formulation are preferably stored at controlled room temperature (15-30° C.).

For administration by inhalation, the compounds for use according to the present invention may be conveniently delivered in the form of an aerosol spray by any number of existing techniques. For example, an aerosol spray may utilize a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch. Alternatively, the compounds may be delivered in aerosol form, absent a propellant.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the compounds of the invention is a cosolvent system comprising benzyl alcohol, a nonpolar surfactant, a water-miscible organic polymer and an aqueous phase such as the VPD cosolvent system. VPD is a solution of 3% w/v benzyl alcohol, 8% w/v of the nonpolar surfactant polysorbate 80, and 65% w/v polyethylene glycol 300, made up to volume in absolute ethanol. The VPD cosolvent system (VPD:D5W) consists of VPD diluted 1:1 with a 5% dextrose in water solution. This cosolvent system dissolves compounds well, and itself produces low toxicity upon systemic administration. Naturally, the proportions of such a cosolvent system may be varied considerably without destroying its solubility and toxicity characteristics. Furthermore, the identity of the cosolvent components may be varied: for example, other low-toxicity nonpolar surfactants may be used instead of polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the JAK2 protein kinase-modulating compounds of the invention may be provided as physiologically acceptable salts wherein the compound may form the negatively or the positively charged species. Examples of salts in which the compound forms the positively charged moiety include, without limitation, salts such as the hydrochloride, sulfate, carbonate, lactate, tartrate, malate, maleate and succinate salts. Salts in which a compound of this invention forms the negatively charged species include, without limitation, the sodium, potassium, calcium and magnesium salts.

Pharmaceutical compositions suitable for use in the present invention include compositions wherein the active ingredients are contained in an amount sufficient to achieve the intended purpose, e.g., the modulation of JAK2 protein kinase activity and/or the treatment or prevention of a JAK2 protein kinase-related disorder.

More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein. For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the protein kinase activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., GOODMAN & GILMAN'S THE PHARMACOLOGICAL BASIS OF THERAPEUCS, Ch. 3, 9^{th} ed., Ed. by Hardman, J., and Limbard, L., McGraw-Hill, New York City, 1996, p. 46.)

Dosage amount and interval may be adjusted individually to provide plasma levels of the active species which are sufficient to maintain the JAK2 kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations. Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of the present invention may range from approximately 2.5 mg/m² to 1500 mg/m² per day. Additional illustrative amounts range from 0.2-1000 mg/qid, 2-500 mg/qid, and 20-250 mg/qid.

In cases of local administration or selective uptake, the effective local concentration of the drug may not be related to plasma concentration, and other procedures known in the art may be employed to determine the correct dosage amount and interval.

The amount of a composition administered will, of course, be dependent on the subject being treated, the severity of the affliction, the manner of administration, the judgment of the prescribing physician, etc.

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

As mentioned above, the compounds and compositions of the invention will find utility in a broad range of diseases and conditions mediated by JAK2 protein kinases. Such diseases may include by way of example and not limitation, Castleman's disease, atherosclerosis, coronary artery disease, peripheral edema, peripheral vascular disease, glaucoma, and wet or dry age-related macular degeneration (AMD), asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration.

Diseases or conditions of humans or other species which can be treated with inhibitors of cytokine or chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid-arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with modulators of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum). In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

The methods of the present invention are accordingly useful in the prevention and treatment of a wide variety of inflammatory and immunoregulatory disorders and diseases, allergic conditions, atopic conditions, as well as autoimmune pathologies. In a specific embodiment, the present invention is directed to the use of the subject compounds for the prevention or treatment of autoimmune diseases, such as rheumatoid arthritis or psoriatic arthritis

The subject treated in the present methods is a mammal, preferably a human being, male or female, in whom modulation of cytokine receptor activity is desired. “Modulation” as used herein is intended to encompass antagonism, agonism, partial antagonism, inverse agonism and/or partial agonism. In a preferred aspect of the present invention, modulation refers to antagonism of cytokine receptor activity. The term “therapeutically effective amount” means the amount of the subject compound that will elicit the biological or medical response of a tissue, system, animal or human that is being sought by the researcher, veterinarian, medical doctor or other clinician

The inventive compound can be used in combination with one or more other chemotherapeutic agents. The dosage of the inventive compounds may be adjusted for any drug-drug reaction. Combined therapy to modulate chemokine receptor activity and thereby prevent and treat inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities

For example, in the treatment or prevention of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, embrel, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

Examples of other active ingredients that may be combined with a compound of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96/40781, WO97/03094, WO97/02289, WO98/42656, WO98/53814, W98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumnic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3, CXCR-3 and CCR-5; (j) cholesterol lowering agents such as HMG-CoA reductase inhibitors (lovastatin, simvastatin and pravastatin, fluvastatin, atorvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), .alpha.-glucosidase inhibitors (acarbose) and glitazones (troglitazone and pioglitazone); (1) preparations of interferon beta (interferon beta-1.alpha., interferon beta-1.beta.); (m) other compounds such as 5-aminosalicylic acid and prodrugs thereof, antimetabolites such as azathioprine and 6-mercaptopurine, and cytotoxic cancer chemotherapeutic agents.

Examples of useful COX-II inhibitors include VIOXX, CELEBREX (celecoxib), valdecoxib, paracoxib, rofecoxib, and Cox 189.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

EGFR-inhibiting agents that may be used in combination include, but are not limited to, the monoclonal antibodies C225 and anti-EGFR 22Mab (ImClone Systems, Inc., New York, N.Y.), the compounds erlotinib (TARCEVA), ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

These and other EGFR-inhibiting agents can be used in the present invention. VEGF inhibitors may be used also in combination in the present method include, for example SU-5416 and SU-6668 (Sugen Inc., South San Francisco, Calif.). VEGF inhibitors are described in, for example, WO 01/60814 A3 (published Aug. 23, 2001), WO 99/24440 (published May 20, 1999), PCT International Application PCT/IB99/00797 (filed May 3, 1999), WO 95/21613 (published Aug. 17, 1995), WO 99/61422 (published Dec. 2, 1999), U.S. Pat. No. 5,834,504 (issued Nov. 10, 1998), WO 01/60814, WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc., Kirkland, Wash.); AVASTIN and LUCENTIS, anti-VEGF monoclonal antibodies of Genentech, Inc.; and angiozyme, a synthetic ribozyme from Ribozyme (Boulder, Colo.) and Chiron (Emeryville, Calif.). These and other VEGF inhibitors can be used in the present invention as described herein. pErbB2 receptor inhibitors, such as GW-282974 (Glaxo Wellcome p1c), and the monoclonal antibodies AR-209 (Aronex Pharmaceuticals Inc., The Woodlands, Tex.) and 2B-1 (Chiron), can furthermore be combined with an inventive compound, for example, those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Pat. No. 6,284,764 (issued Sep. 4, 2001), incorporated in its entirety herein by reference. The erbB2 receptor inhibitor compounds and substance described in the aforementioned PCT applications, U.S. patents, and U.S. provisional applications, as well as other compounds and substances that inhibit the erbB2 receptor, can be used with an inventive compound, in accordance with the present invention.

The invention will be further understood upon consideration of the following non-limiting Examples.

TABLE 1
Representative Pyrimidine-2,4-Diamine Derivatives

| Compound | |
|---|---|
| No. | Structure |
| 1-1 | 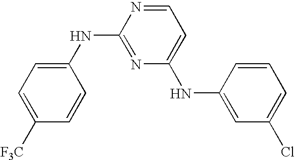 |
| | Chemical Formula: C₁₇H₁₂ClF₃N₄ |
| | Molecular Weight: 364.75 |
| 1-2 | 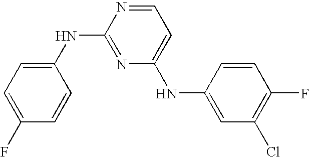 |
| | Chemical Formula: C₁₆H₁₁ClF₂N₄ |
| | Molecular Weight: 332.74 |
| 1-3 | 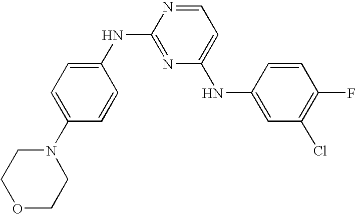 |
| | Chemical Formula: C₂₀H₁₉ClFN₅O |
| | Molecular Weight: 399.85 |
| 1-4 | 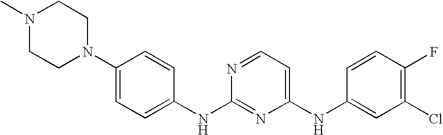 |
| | Chemical Formula: C₂₁ H₂₂ClFN₆ |
| | Molecular Weight: 412.89 |
| 1-5 | 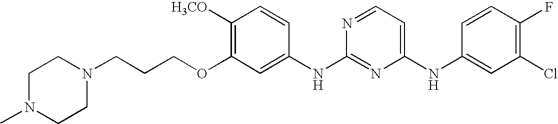 |
| | Chemical Formula: C₂₅H₃₀ClFN₆O₂ |
| | Molecular Weight 501.00 |
| 1-6 | 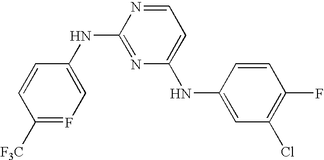 |
| | Chemical Formula: C₁₆H₁₀ClF₄N₅ |
| | Molecular Weight: 383.73 |
| 1-7 | 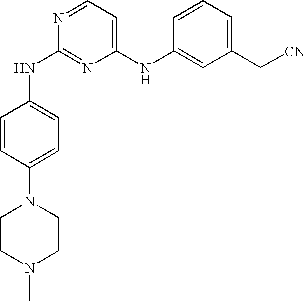 |
| | Chemical Formula: C₂₃H₂₅N₇ |
| | Molecular Weight: 399.49 |
| 1-8 | 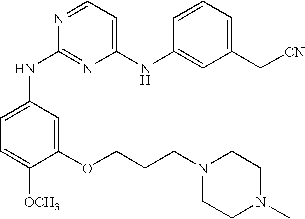 |
| | Chemical Formula: C₂₇H₃₃N₇O₂ |
| | Molecular Weight: 487.60 |
| 1-9 | 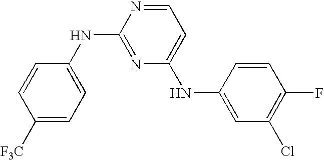 |
| | Chemical Formula: C₁₇H₁₁ClF₄N₄ |
| | Molecular Weight: 382.74 |
| 1-10 | 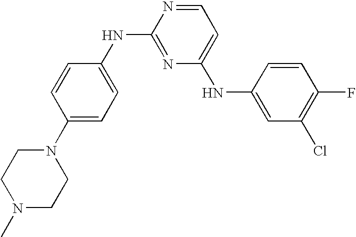 |
| | Chemical Formula: C₂₂H₂₂FN₇ |
| | Molecular Weight: 403.46 |
| 1-11 | 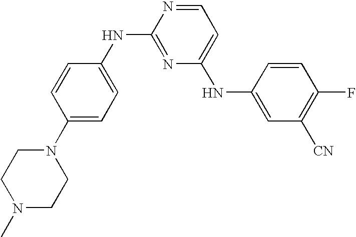 |
| | Chemical Formula: C₂₃H₂₄FN₇ |
| | Molecular Weight: 417.48 |
| 1-12 | 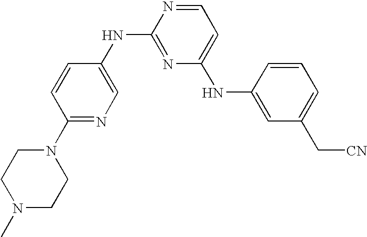 |
| | Chemical Formula: C₂₂H₂₄N₆ |
| | Molecular Weight: 400.48 |
| 1-13 | 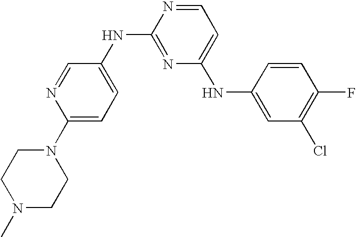 |
| | Chemical Formula: C₂₀H₂₁ClFN₇ |
| | Molecular Weight: 413.88 |
| 1-14 | 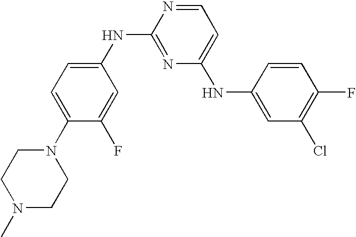 |
| | Chemical Formula: C₂₁ H₂₁ClF₂N₆ |
| | Molecular Weight: 430.88 |
| 1-15 | 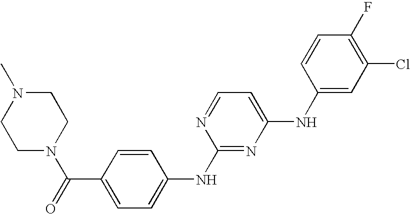 |
| | Chemcal Formula: C₂₂H₂₂ClFN₆O |
| | Molecular Weight: 440.90 |
| 1-16 | 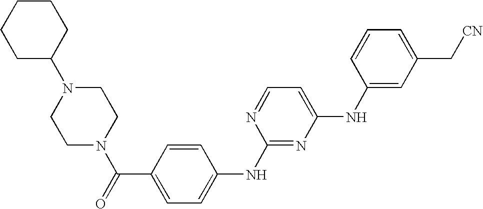 |
| | Chemical Formula: C₂₉H₃₃N₇O |
| | Molecular Weight: 495.62 |
| 1-17 | 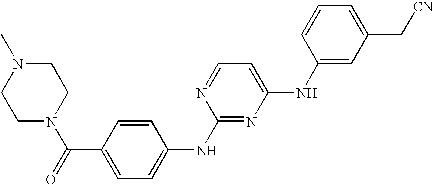 |
| | Chemical Formula: C₂₄H₂₅N₇O |
| | Molecular Weight: 427.50 |
| 1-18 | 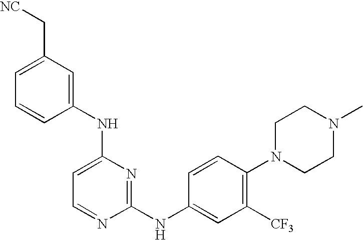 |
| | Chemical Formula: C₂₄H₂₄F₃N₇ |
| | Molecular Weight: 467.49 |

### General Synthesis of Pyrimidine-2,4-Diamine Derivatives

Compounds of the invention may be made by one of ordinary skill in the chemical arts using conventional synthetic procedures, as well as by the following general reaction scheme.

General Reaction Scheme:

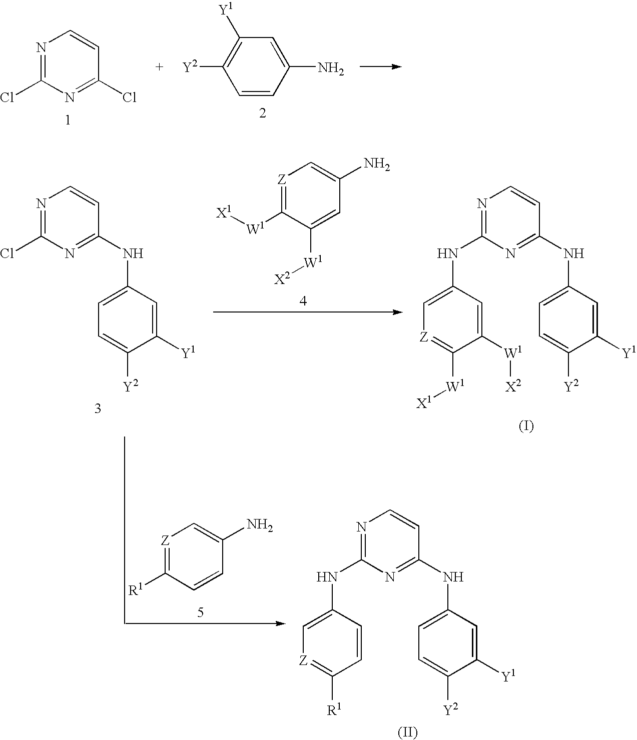

In brief, 2,4-dichloropyrimidine (1) is reacted with an appropriately substituted aniline (2) to yield intermediate (3). Intermediate (3) is then reacted with a further appropriately substituted amine (4) to yield a compound of structure (I). Alternatively, intermediate (3) is further reacted with an appropriately substituted amine (5) to yield a compound of structure (II). Without specific statement, all solvents and reagents are available from Aldrich or VWR chemicals and may be used as supplied or purified by standard laboratory methods as required.

### Synthesis of Pyrimidine-2,4-Diamine Derivatives

A. General Procedure to Prepare 2-Chloro-4-Substitued Pyrimidine (3)

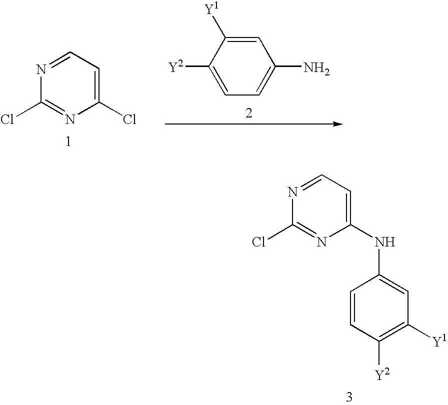

A reaction mixture containing 2,4-dichloropyrimidine (1) (10 mmol), aniline (2) (10 mmol) and Diisopropylethyl amine (DIPEA) (1.5 mmol) in 30 mL of isopropanol (IPA) was refluxed overnight. The solvent was removed and the residue was purified with using a Combiflash Companion system (10% to 70% Hexane/EtOAc) to give the desired 2-chloro-4-substitued pyrimidine (3).

### 2-Chloro-N-(3-chloro-4-fluorophenyl)pyrimidin-4-amine (7)

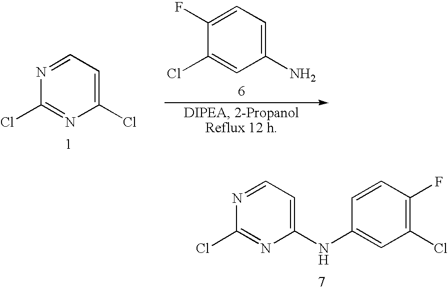

A reaction mixture of 2,4-dichloropyrimidine (1) (1 g, 6.71 mmol, Sigma-Aldrich), 3-chloro-4-fluoroaniline (6) (0.977 g, 6.71 mmol, Sigma-Aldrich) and diisopropylethylamine (DIPEA) (3.47 g, 26.8 mmol) in 2-propanol (20 mL) was refluxed for 18 h. The solvent was evaporated and purified by Combiflash Companion using 70% Hexane to pure dichloromethane (DCM) and 20% ethylacetate (EtOAc) solvent system (40 g normal phase RediSep Flash column with run time 50 min) to afford compound (7) (0.8 g). (TLC, Rf=0.6, EtOAc). White solid, yield 46.2%. ¹H-NMR (400 MHz, DMSO-d₆) 10.18 (s, 1H), 8.20 (d, J=6.2 Hz, 1H), 7.90 (m, 1H), 7.50 (m, 1H), 7.43 (t, J=8.9 Hz, 1H), 6.74 (d, J=5.8 Hz, 1H). ESI/MS m/z 258.0 (100%), 259.9 (78%), 261.9 (18%).

### 5-(2-Chloropyrimidin-4-ylamino)-2-fluorobenzonitrile (9)

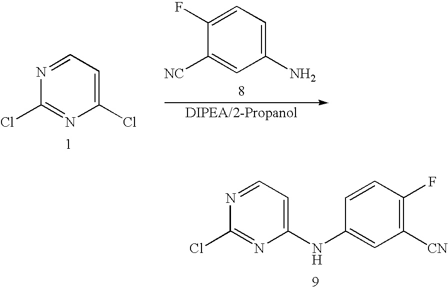

A reaction mixture of 2,4-dichloropyrimidine (1) (1 g, 6.71 mmol), 5-amino-2-fluorobenzonitrile (8) (0.914 g, 6.71 mmol) in 2-propanol (50 mL) and 1 mL of DIPEA was refluxed overnight. The solvent was evaporated and the crude material was purified by Combiflash (12 g, DCM to 10% MeOH/DCM) to afford 318 mg of compound (9) (TLC, Rf=0.1, 6% MeOH/DCM). White solid, yield 19%. ¹H NMR (400 MHz, CD₃OD) 8.15 (m, 2H), 7.89 (m, 1H), 7.35 (t, J=9.23 Hz, 1H), 6.70 (d, J=5.6 Hz, 1H). ESI/MS m/z 248.9 (100%), 250.9 (33%).

### 2-(3-aminophenyl)acetonitrile (11)

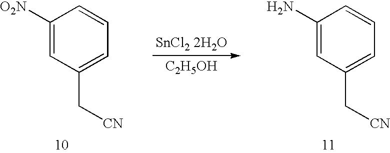

3-Nitrophenyl acetonitrile (10) was dissolved in a mixture of concentrated HCl (15 ml) and ethanol (15 ml). A solution of SnCl₂ dihydrate in ethanol (25 ml) was added gradually dropwise under ice-cooling and the mixture was stirred at room temperature for 5 hours. TLC (EtOAc/Hexane 1:1) Rf=0.1 showed 80% completion. After the solvent was evaporated, the residue was treated with NaOH to pH 9. Extraction with EtOAc and combiflash (12 g, Hexane/EtOAc 10% to 50%, 70 min) afforded 1.182 g of compound (11) as a yellow oil. ¹H-NMR (400 Mhz, CDCl₃) 7.12 (t, J=7.5 Hz, 1H), 6.65 (m, 3H), 3.72 (br-s, 1H), 3.64 (s, 2H).

### 2-(3-(2-Chloropyrimidin-4-ylamino)phenyl)acetonitrile (12)

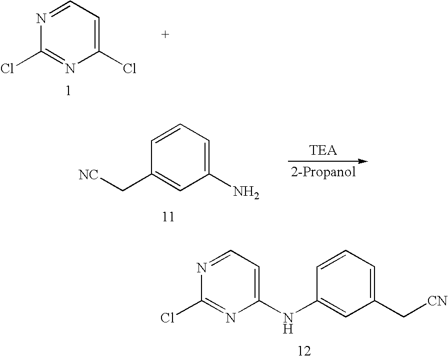

A reaction mixture of 2,4-dichloropyrimidine (1) (1.127 g, 7.57 mmol) 2-(3-aminophenylacetonitrile (11) (1 g, 7.57 mmol) in 2-propanol (40 mL) and 15 mmol of triethylamine (TEA) was refluxed for 2 days. The solvent was evaporated and purified by Combiflash (12 g, twice, CHCl₃) to afford 932 mg of compound (12) as pale-green crystals. Yield 50%. ¹H-NMR (400 MHz, CDCl₃) 8.16 (d, J=6.15 Hz, 1H), 7.41 (m, 1H), 7.33 (m, 2H), 7.18 (d, J=7.5 Hz, 1H), 6.96 (br-s, 1H), 6.58 (d, J=5.8 Hz, 1H), 3.77 (s, 2H).

B. General Procedure to Prepare 2,4-Disubstituted Pyrimidine (I)

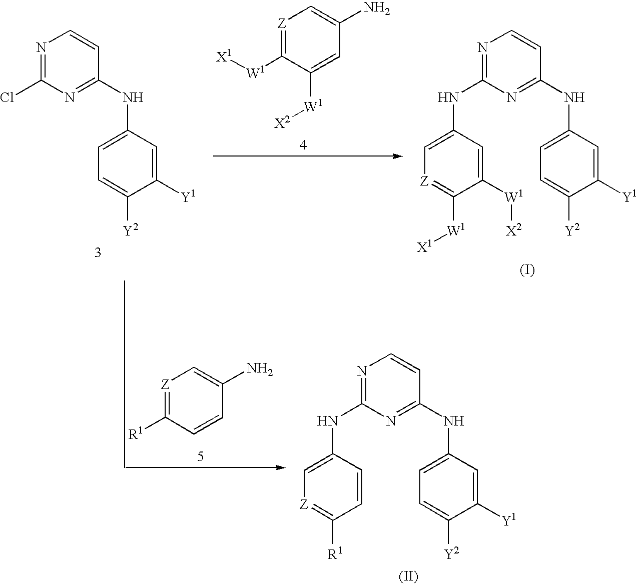

A reaction mixture containing 2-chloro-4-substituted pyrimidine (3) (0.25 mmol), amine (4) or amine (5) (Apollo Scientific and Bionet Building Blocks, UK) (0.25 mmol) and TFA (0.5 mmol) in 7 mL of isopropanol was refluxed overnight. NaOH (0.6 mmol) was added after the reaction mixture was cooled down to room temperature. The solvent was removed and the residue was purified by Combiflash (4 g, DCM to 20% MeOH/DCM) to give the compounds of Structure (I) or (II) as noted below.

### N4-(3-chlorophenyl)-N2-(4-(trifluoromethyl)phenyl)pyrimidine-2,4-diamine (Compound 1-1)

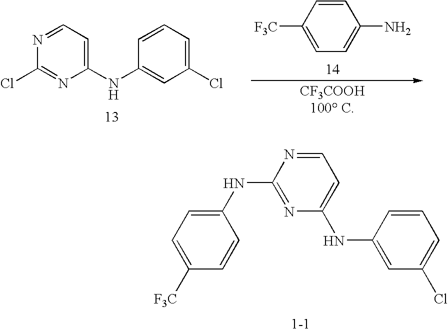

To a solution of 2-chloro-N-(3-chlorophenyl)pyrimidin-4-amine (13) (100 mg, 0.417 mmol) in 2-propanol (10 mL) was added 4-(trifluoromethyl)aniline (14) (67.1 mg, 0.417 mmol) followed by addition of catalytic trifluoroacetic acid and stirring at reflux temperature overnight. The solvent was evaporated and the residue was purifed by Combiflash Companion using a DCM and 5% MeOH solvent system (4 g normal phase RediSep Flash column with run time 50 min at flow 18 ml/min) to afford Compound 1-1. (TLC, Rf=0.4, 10% MeOH/DCM). ¹H NMR (400 MHz, DMSO-d₆) 8.08 (d, J=6.15 Hz, 1H), 7.8 (s, 2H), 3.39 (m, 1H), 7.82 (d, J=8.2 Hz, 2H), 7.66 (d, J=8.2 Hz, 2H), 7.46 (d, J=6.84 Hz, 1H), 7.36 (t, J=8.2 Hz, 1H), 7.15 (d, J=8.18 Hz, 1H), 6.42 (d, J=6.15 Hz, 1H) ESI/MS m/z 365.0 (M+H)⁺.

### N4-(3-chloro-4-fluorophenyl)-N2-(4-fluorophenyl)pyrimidine-2,4-diamine (Compound 1-2)

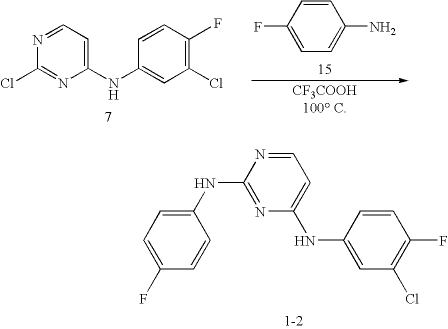

To a solution of 2-chloro-N-(3-chloro-4-fluorophenyl)pyrimidin-4-amine (7) (60 mg, 0.232 mmol) in 2-propanol (10 mL) was added 4-fluoroaniline (15) (25.8 mg, 0.232 mmol) followed by the addition of catalytic trifluoroacetic acid and stirring at reflux temperature overnight. To the reaction mixture was added Triethylamine followed by stirring for 1 h before TLC showed completion of the reaction. The solvent was evaporated and the residue purified by Combiflash Companion using Hexane 70% and DCM solvent system (4 g normal phase RediSep Flash column with run time 50 min at flow 18 ml/min) to afford Compound 1-2. (TLC, Rf=0.35, 5% MeOH/DCM). ¹H NMR (400 MHz, DMSO-d₆) 8.01 (m, 2H), 7.64 (m, 2H), 3.39 (m, 2H), 7.49 (m, 1H), 7.35 (t, J=8.23 Hz, 1H), 7.11 (m, 2H), 6.22 (m, 1H), ESI/MS m/z 333.0 (M+H)⁺.

### N4-(3-chloro-4-fluorophenyl)-N2-(4-morpholinophenyl)pyrimidine-2,4-diamine (Compound 1-3)

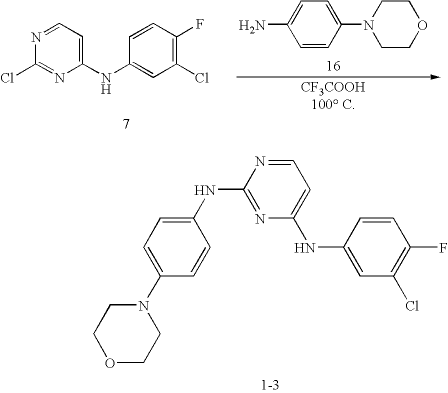

To a solution of 2-chloro-N-(3-chloro-4-fluorophenyl)pyrimidin-4-amine (7) (60 mg, 0.232 mmol) in 2-propanol (10 mL) was added 4-morpholinoaniline (16) (41.4 mg, 0.232 mmol) followed by the addition of catalytic trifluoroacetic acid and stirring at reflux temperature overnight. The crude residue was purified by CombiFlash Companion using 10% MeOH in DCM solvent system (4 g normal phase RediSep Flash column with run time 40 min at flow 26 mL/min) to obtain compound 1-3. (TLC, Rf=0.45, 10% MeOH/DCM) HPLC=94%. ¹HNMR (400 MHz, DMSO-d₆) 8.03 (m, 1H), 7.90 (m, 1H), 3.39 (m, 2H), 7.31 (d, J=8.54 Hz, 2H), 6.98 (d, J=8.55 Hz, 2H), 6.33 (d, J=6.83 Hz, 1H), 3.74 (m, 4H), 3.10 (s, 4H) ESI/MS m/z 400.1 (M+H)⁺.

### N4-(3-chloro-4-fluorophenyl)-N2-(4-(4-methylpiperazin-1-yl)phenyl)pyrimidine-2,4-diamine (Compound 1-4)

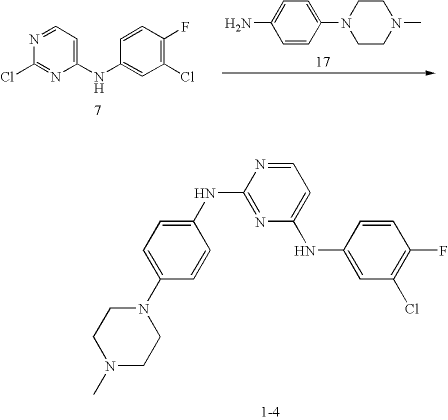

2-chloro-N-(3-chloro-4-fluorophenyl)pyrimidin-4-amine (7) (100 mg, 0.387 mmol), 4-(4-methylpiperazine-1-yl)aniline (17) (74.1 mg, 0.387 mmol, purchased from Apollo Scientific, UK), DIPEA (2 mL, 11.48 mmol), and dimethylaminopyridine (DMAP) in 1-butanol was refluxed overnight. TLC (6% MeOH/DCM) showed complete reaction. Concentration and CombiFlash purification (4 g, DCM to 100% EtOAc) afforded 7 mg of compound 1-4., which is a purple color under phosphomolybdic acid stain. Yield 48%. ¹H-NMR (400 MHz, DMSO-d₆) 9.45 (s, 1H), 8.98 (s, 1H), 8.08 (m, 1H), 7.98 (d, J=4.5 Hz, 1H), 7.45 (m, 3H), 7.30 (t, J=9.2 Hz, 1H), 6.86 (d, J=8.9 Hz, 2H), 6.10 (d, J=5.8 Hz, 1H), 3.04 (m, 4H), 2.43 (m, 4H), 2.20 (s, 3H). ESI/MS m/z 413.3 (100%), 415.2 (33%).

### N4-(3-chloro-4-fluorophenyl)-N2-(4-methoxy-3-(3-(4-methylpiperazin-1-yl)propoxy)phenyl)pyrimidine-2,4-diamine (Compound 1-5)

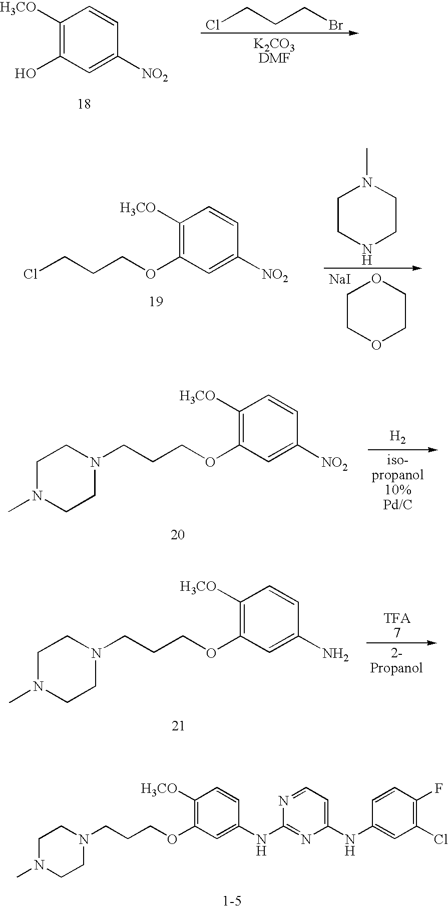

To a mixture of 2-methoxy-5-nitrophenol (18) (2 g, 11.82 mmol), 3-chloropropyl bromide (2.234 g, 23.65 mmol) in dimethylformamide (DMF) (20 mL) was added K₂CO₃ (3.27 g) and the resulting reaction mixture was heated to 80° C. for 3 h. TLC (EtOAc) showed that the reaction was complete. The reaction mixture was then partitioned between water (100 mL) and ethyl acetate (150 mL). The organic layer was washed with saturated NaHCO₃ and water, then dried over MgSO₄ and filtered. The solution was concentrated to afford 19 as a pale yellow solid (3.9 g). To 19 (1.5 g, 6.11 mmol) was added N-methylpiperazine in 20 mL of 1,4-dioxane and sodium iodide (1.373 g, 9.16 mmol), and the resulting reaction mixture was heated to 100° C. for 30 hrs. The orange mixture was concentrated to remove 1,4-dioxane and then diluted with 50 mL of ethyl acetate. After stirring for 0.5 h, it was filtered to remove salt. The organic layer was washed with NaOH solution (20 mL) and water (3×20 mL). Drying over MgSO₄ and concentration to gave compound 20 as an orange oil (1.00 g). ¹H-NMR (400 MHz, CDCl₃) 7.90 (dd, J1=2.3 Hz, J2=8.9 Hz, 1H), 7.75 (d, J=2.3 Hz, 1H), 6.88 (d, J=8.8 Hz, 1H), 4.14 (m, 2H), 3.94 (s, 3H), 2.58 (m, 10H), 2.43 (br-s, 3H), 2.03 (m, 2H).

Compound 20 was subjected to hydrogenation in the presence of 10% Pd/C in isopropanol in a Parr shaker for 5 hrs. TLC (15% MeOH/DCM) showed reaction completion. Filtration through celite and concentration afforded compound 21 (1 g) as a brown oil. ¹H-NMR (400 MHz, CDCl₃) 6.68 (d, J=8.2 Hz, 1H), 6.30 (d, J=2.4 Hz, 1H), 6.20 (dd, J₁=2.4 Hz, J2=8.2 Hz, 1H), 3.99 (t, J=6.5 Hz, 2H), 3.75 (s, 3H), 3.46 (s, 3H), 2.55 (br-m, 10H), 2.32 (s, 3H), 2.01 (m, 2H).

A reaction mixture containing 21 (100 mg, 0.387 mmol) and 7 (108 mg, 0.387 mmol) in TFA (1 mL) and 2-propanol (10 mL) was refluxed overnight. TLC (6% DCM/MeOH) showed the complete consumption of 7. The reaction mixture was concentrated and redissolved into MeOH. TFA was quenched with the addition of NaOH. Concentration and CombiFlash purification (4 g, DCM to 15% MeOH/DCM) afforded 219 mg of Compound 1-5 as a brown foam. ¹H-NMR (400 MHz, DMSO-d₆) 9.49 (s, 1H), 9.01 (s, 1H), 8.04 (m, 1H), 8.00 (d, J=5.8 Hz, 1H), 7.4 (br-s, 1H), 7.29 (t, J=9.3 Hz, 1H), 7.20 (m, 2H), 6.85 (d, J=8.8 Hz, 1H), 6.13 (d, J=5.8 Hz, 1H), 3.86 (t, J=6.15 Hz, 2H), 3.88 (s, 3H), 2.48 (s, 8H), 2.35 (s, 3H), 1.83 (m, 2H). ESI/MS m/z, 501.2 (100%), 503.3 (33%).

### N4-(3-chloro-4-fluorophenyl)-N2-(6-(trifluoromethyl)pyridin-3-yl)pyrimidine-2,4-diamine (Compound 1-6)

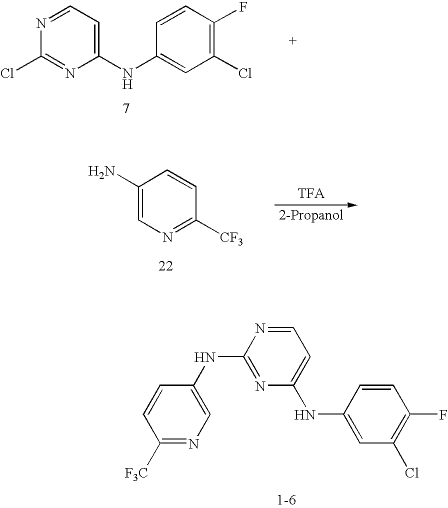

To a mixture of 7 (100 mg, 0.387 mmol) and 5-amino-2-(trifluoromethyl)pyridine (22) (62.8 mg, 0.387 mmol) in 2-propanol (10 mL) was added TEA and the reaction mixture was refluxed for 24 h. TLC (EtOAc, Rf=0.3) showed reaction completion. The mixture was concentrated to remove isopropanol and redissolved into methanol. NaOH was added to neutralize TFA. Further concentration and CombiFlash purification (4 g, Hexane/EtOAc 15% to 90%, 50 min) afforded 106 mg of Compound 1-6 as a solid. ¹H-NMR (400 MHz, CDCl₃) 8.69 (s, 1H), 8.42 (d, J=8.5 Hz, 1H), 8.11 (d, J=5.4 Hz, 1H), 7.59 (m, 1H), 7.53 (m, 1H), 7.15 (m, 3H), 6.49 (s, 1H), 6.19 (d, J=5.5 Hz, 1H). ESI/MS m/z, 384.0 (100%), 386.0 (33%)

### 2-(3-(2-(4-(4-Methylpiperazin-1-yl)phenylamino)pyrimidin-4-ylamino)phenyl)acetonitrile (Compound 1-7)

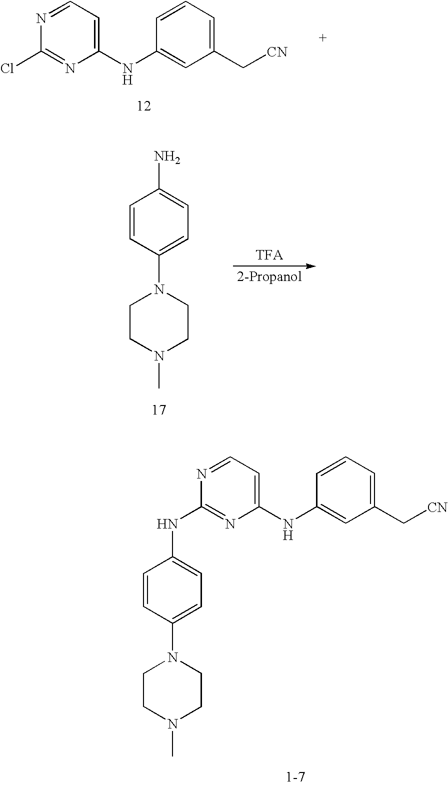

Compounds 12 (100 mg, 0.409 mmol) and 17 (78 mg, 0.409 mmol, Apollo Scientific, UK) were dissolved in 10 mL of 2-propanol and TEA (1 mL) was added. The resulting reaction mixture was refluxed overnight. TLC (20% MeOH/DCM, Rf=0.2) showed reaction complete. NaOH was added and some white precipitate formed. Concentration and CombiFlash purification (0˜25% MeOH/DCM) afforded Compound 1-7 (186 mg) as a white solid. ¹HNMR indicated maybe some aniline inside. Yield 114%. ¹H-NMR (400 MHz, CD₃OD) 7.88 (d, J=6.1 Hz, 1H), 7.79 (s, 1H), 7.44 (m, 3H), 7.27 (t, J=7.86 Hz, 1H), 7.00 (m, 3H), 6.15 (d, J=6.1 Hz, 1H), 3.75 (s, 2H), 3.27 (m, 4H), 2.96 (m, 4H), 2.59 (s, 3H). ESI/MS m/z, 400.1 (100%).

### 2-(3-(2-(4-Methoxy-3-(3-(4-methylpiperazin-1-yl)propoxy)phenylamino) pyrimidin-4-ylamino)phenyl)acetonitrile (Compound 1-8)

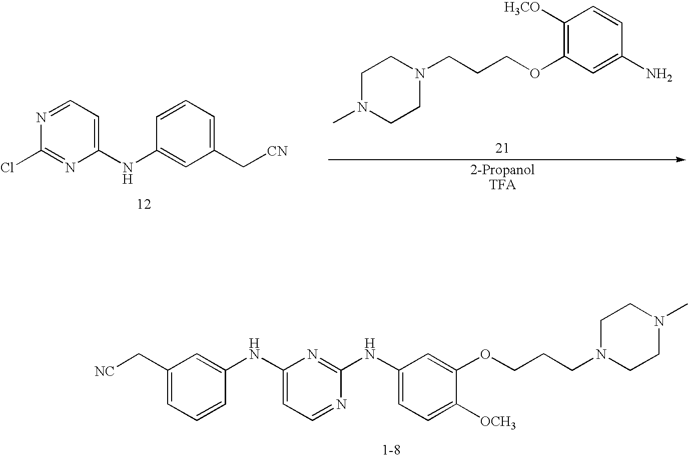

To a mixture of 12 (100 mg, 0.409 mmol) and 21 (114 mg, 0.409 mmol) in 2-propanol (10 mL) was added TFA, and it was refluxed overnight. TLC (20% MeOH/DCM, Rf=0.1) showed reaction completion. Addition of NaOH resulted in a white precipitate. Concentration and Combiflash purification (0 to 25% MeOH:DCM) afforded Compound 1-8 (236 mg) as a solid foam. ¹H-NMR (400 MHz, CD₃OD): 7.90 (d, J=5.8 Hz, 1H), 7.78 (s, 1H), 7.45 (d, J=8.2 Hz, 1H), 7.26 (m, 2H), 6.98 (m, 3H), 6.16 (d, J=6.2 Hz, 1H), 3.91 (t, J=5.8 Hz, 2H), 3.84 (s, 3H), 3.74 (s, 2H), 3.34 (s, 2H), 2.71 (br-m, 2H), 2.56 (s, 3H), 1.96 (m, 2H). ESI/MS m/z, 488.2 (100%).

### N4-(3-chloro-4-fluorophenyl)-N2-(4-(trifluoromethyl)phenyl)pyrimidine-2,4-diamine (Compound 1-9)

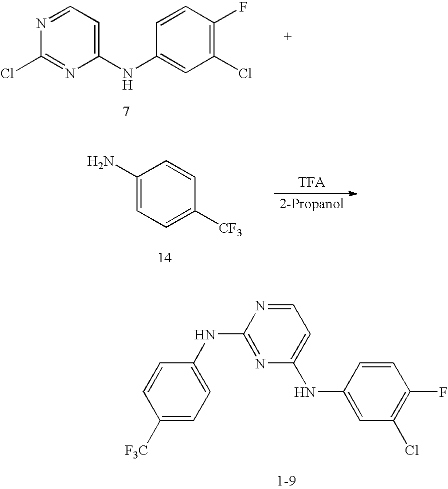

To a mixture of 7 (60 mg, 0.232 mmol) and 14 (37.5 mg, 0.232 mmol) in 2-propanol (7 mL) was added TFA, and it was refluxed overnight. TLC (6% MeOH/DCM, Rf=0.15) showed reaction completion. NaOH was added to neutralize the TFA. Concentration of the crude and CombiFlash purification (4 g, 0 to 10% MeOH/DCM, 50 min) afforded Compound 1-9 (70 mg) as a white solid. ¹H NMR (400 MHz, CD₃OD) 7.99 (d, J=5.8 Hz, 1H), 7.95 (dd, J₁=2.7 Hz, J₂=6.8 Hz, 1H), 7.79 (d, J=8.5 Hz, 2H), 7.53 (d, J=8.5 Hz, 2H), 7.44 (m, 1H), 7.17 (t, J=8.9 Hz, 1H), 6.23 (d, J=5.8 Hz, 1H). ESI/MS m/z, 383.1 (100%), 385.1 (33%).

### 2-Fluoro-5-(2-(4-(4-methylpiperazin-1-yl)phenylamino)pyrimidin-4-ylamino) benzonitrile (Compound 1-10)

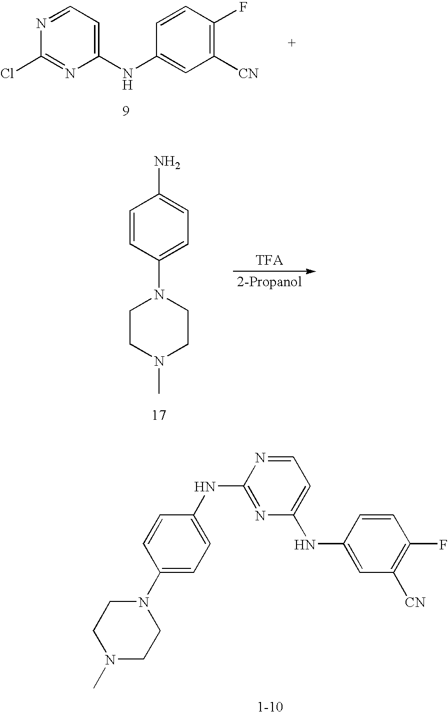

A mixture of 9 (60 mg, 0.241 mmol) and 17 (46.2 mg, 0.241 mmol), Apollo Scientific, UK) were dissolved in 10 mL of 2-propanol and catalytic TFA was added. The resulting mixture was refluxed overnight. TLC (20% MeOH/DCM, Rf=0.1) showed reaction complete. NaOH was added to neutralize TFA. Concentration and CombiFlash purification (4 g normal phase RediSep Flash column with run time 40 min at flow 26 mL/min, 0˜20% MeOH/DCM) afforded Compound 1-10 (82 mg, 84%) as white solid. ¹H NMR (400 MHz, CD₃OD) 8.35 (m, 1H), 7.92 (d, J=5.8 Hz, 1H), 7.70 (m, 1H), 7.40 (d, J=8.9 Hz, 2H), 7.23 (t, J=9.8 Hz, 1H), 7.01 (d, J=9.2 Hz, 2H), 6.12 (d, J=5.8 Hz, 1H), 3.23 (m, 4H), 2.70 (br-s, 4H), 2.40 (s, 3H). ESI/MS m/z 404.1 (M+H)

### 2-(3-(2-(3-Fluoro-4-(4-methylpiperazin-1-yl)phenylamino)pyrimidin-4-ylamino)phenyl)acetonitrile (Compound 1-11)

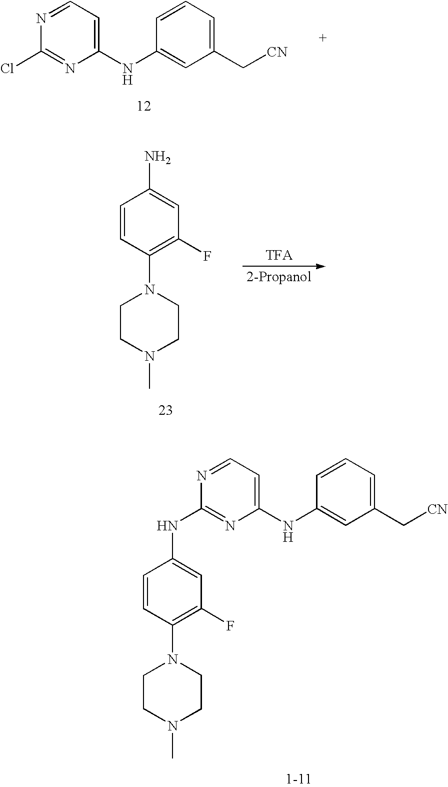

A mixture of 12 (60 mg, 0.245 mmol) and 3-fluoro-4-(4-methylpiperazine-1-yl)aniline (23) (51.3 mg, 0.245 mmol, purchased from Apollo Scientific, UK) in 2-propanol (7 mL) and catalytic TFA was refluxed overnight. TLC (20% MeOH/DCM, Rf=0.2) showed reaction completion. Addition of NaOH yielded a white precipitate. Concentration and CombiFlash purification (4 g, 0˜20% MeOH/DCM) afforded Compound 1-11 (115 mg) as a white solid. ¹H NMR (400 MHz, CD₃OD) 7.93 (d, J=6.1 Hz, 1H), 7.73 (s, 1H), 7.61 (dd, J₁=2.4 Hz, J₂=4.7 Hz, 1H), 7.53 (d, J=7.8 Hz, 1H), 7.32 (t, J=8.2 Hz, 1H), 7.20 (d, J=8.9 Hz, 1H), 7.00 (m, 2H), 6.20 (d, J=5.8 Hz, 1H), 3.84 (s, 2H), 3.16 (br-s, 4H), 2.92 (br-s, 4H), 2.56 (s, 3H). ESI/MS m/z 418.1 (M+H).

### 2-(3-(2-(6-(4-Methylpiperazin-1-yl)pyridin-3-ylamino)pyrimidin-4-ylamino)phenyl)acetonitrile (Compound 1-12)

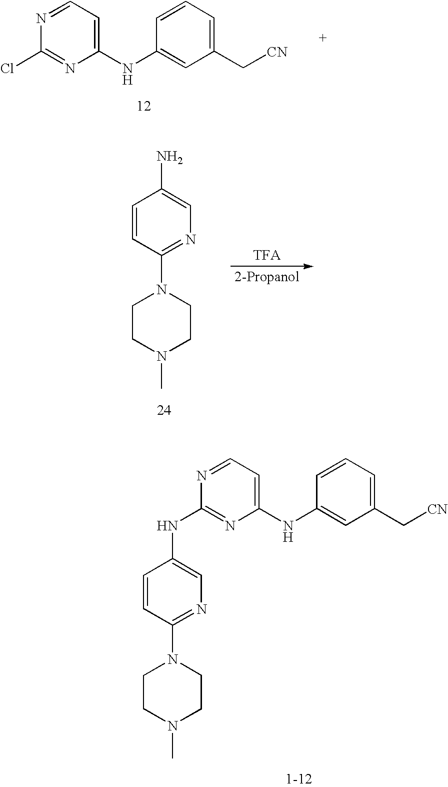

A mixture of 12 (60 mg, 0.245 mmol) and 6-(4-methylpiperazine-1-yl)pyridine-3-amine (24) (47.1 mg, 0.245 mmol, purchased from Bionet Building blocks UK) in 2-propanol (7 mL) and catalytic TFA was refluxed for 24 hrs. TLC (20% MeOH/DCM, Rf=0.1) showed reaction completion. NaOH was added to neutralize TFA. Concentration and CombiFlash purification (4 g, 0˜20% MeOH/DCM) afforded Compound 1-12 (97 mg) as a solid. Yield 99%. ¹H NMR (400 MHz, CD₃OD) 8.35 (d, J=2.7 Hz, 1H), 7.88 (d, J=6.2 Hz, 1H), 7.83 (s, 1H), 7.76 (dd, J₁=2.7 Hz, J₂=9.2 Hz, 1H), 7.37 (d, J=8.5 Hz, 1H), 7.27 (t, J=7.9 Hz, 1H), 7.01 (d, J=7.5 HZ, 1H), 6.88 (d, J=9.2 Hz, 1H), 6.16 (d, J=5.8 Hz, 1H), 3.81 (s, 2H), 3.61 (br-s, 4H), 2.87 (br-s, 4H), 2.56 (s, 3H)

### N4-(3-chloro-4-fluorophenyl)-N2-(6-(4-methylpiperazin-1-yl)pyridin-3-yl)pyrimidine-2,4-diamine (Compound 1-13)

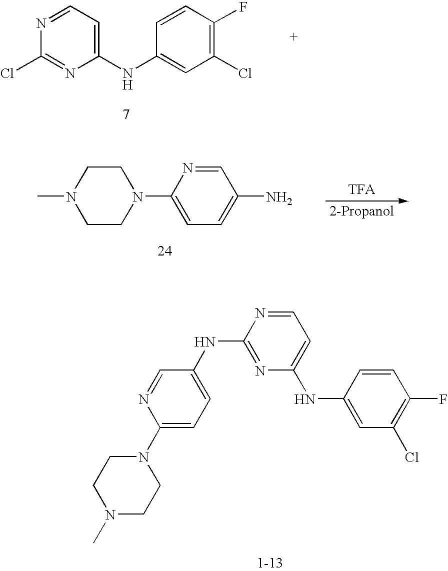

A mixture of 7 (60 mg, 0.232 mmol) and 24 (44.7 mg, 0.232 mmol) in 2-propanol (7 mL) and catalytic TFA was refluxed for 24 hrs. TLC (20% MeOH/DCM) showed reaction completion. NaOH was added, the mixture was concentrated and CombiFlash purification was performed (4 g, 100% DCM to 25% MeOH/DCM) to afford Compound 1-13 (86.7 mg) as a white solid. 90% yield. ¹H NMR (400 MHz, CD₃OD) 8.23 (d, J=2.4 Hz, 1H), 7.92 (m, 1H), 7.90 (d, J=5.8 Hz, 1H), 7.82 (dd, J1=9.2 Hz, J2=2.7 Hz, 1H), 7.35 (m, 1H), 7.12 (t, J=8.9 Hz, 1H), 6.88 (d, J=9.2 Hz, 1H), 3.61 (br-s, 4H), 2.90 (m, 4H), 2.59 (s, 3H) ESI/MS m/z 414.1 (M+H).

### N4-(3-chloro-4-fluorophenyl)-N2-(3-fluoro-4-(4-methylpiperazin-yl)phenyl)pyrimidine-2,4-diamine (Compound 1-14)

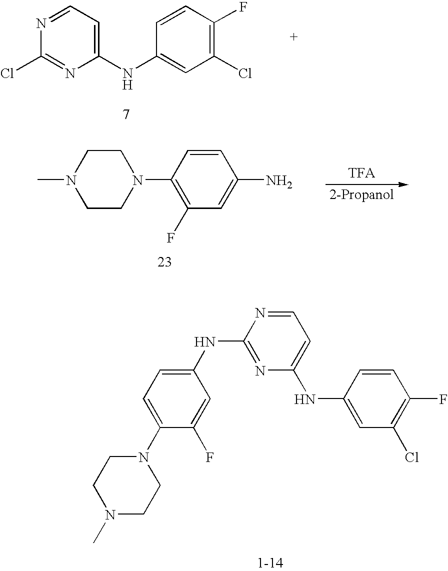

A mixture of 7 (60 mg, 0.232 mmol) and 23 (48.7 mg, 0.232 mmol) in 2-propanol (7 mL) and catalytic TFA was refluxed for 24 hrs. before TLC (20% MeOH/DCM, Rf=0.1) showed reaction completion. After 3 days at room temperature, some white solid was formed (103 mg). Filtration and ¹H NMR indicated that the product was a TFA salt. NaOH was added and the mixture concentrated and purified by CombiFlash companion (4 g, DCM to 25% MeOH) which afforded Compound 1-14 (88.8 mg) as a white solid. The yield was 89%. ¹H NMR (400 MHz, CD₃OD) 7.90 (m, 2H), 7.47 (m, 2H), 7.27 (dd, J1=9.6 Hz, J2=2.4 Hz, 1H), 7.14 (t, J=6.4 Hz, 1H), 6.99 (t, J=9.6 Hz, 1H), 6.15 (d, J=5.8 Hz, 1H), 3.10 (br-s, 4H), 2.75 (br-s, 4H), 2.43 (s, 3H). ESI/MS m/z 431.0 (M+H).

### (4-(4-(3-chloro-4-fluorophenylamino)pyrimidin-2-ylamino)phenyl)(4-methyl piperazin-1-yl)methanone (Compound 1-15)

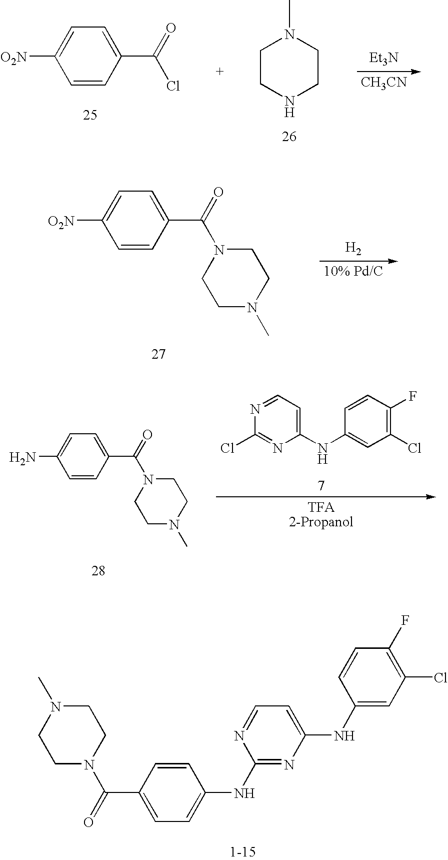

A solution of 4-nitrobenzoyl chloride (25) (1 g, 5.39 mol) in CH₃CN (50 mL) was treated with 1-methylpiperazine (26) at 10° C. over 10 min. The reaction mixture was stirred for another 1 h followed by addition of TEA (1.49 mL, 2 eq) and stirring for an additional half hour. The reaction mixture was diluted with ice-cold water (150 mL) and extracted with EtOAc (4×150 mL). The organic layer was dried over MgSO₄ and concentrated to give 1.1 g (82% yield) (4-methylpiperazin-1-yl)(4-nitrophenyl)methanone 27 as a yellow solid. TLC: 10% MeOH/DCM, Rf=0.6. ¹NMR (400 MHz, CDCl₃) 8.28 (d, J=8.5 Hz, 2H), 7.56 (d, J=8.5 Hz, 2H), 3.88 (br-s, 4H), 3.46 (br-s, 4H), 2.40 (s, 3H) ESI/MS m/z 250.0 (M+H)

A mixture containing (4-methylpiperazin-1-yl)(4-nitrophenyl)methanone (27) (200 mg, 0.802 mmol) and 10% Pd/C (60 mg) in 20 mL of Ethanol was shaken under H₂ (60 psi) for 4 hrs. After reaction completion and filtration, concentration afforded 28 (83.8 mg, yield 48%) as a pale yellow oil. ¹H-NMR (400 Mhz, CD₃OD) 7.19 (dd, J1=2.0 Hz, J2=6.5 Hz, 2H), 6.68 (dd, J1=2.7 Hz, J2=6.5 Hz, 2H), 3.64 (br-s, 4H), 2.44 (br-s, 4H), 2.31 (s, 3H) ESI/MS m/z 220.0 (M+H).

A mixture of 7 (50 mg, 0.194 mmol) and 28 (33 mg, 0.150 mmol) in 2-propanol (5 mL) and catalytic TFA was microwaved for 1 h at 150° C. TLC showed completion of the reaction. HPLC indicated a possible major new spot. CombiFlash purification (4 g, DCM to 15% MeOH/DCM) afforded 32 mg of Compound 1-15 as a semi-solid. ¹H-NMR (400 MHz, CD₃OD) 7.98 (d, J=6.15 Hz, 1H), 7.94 (m, 1H), 7.72 (d, J=8.54, 2H), 7.36 (dd, J1=2.0 Hz, J2=6.8 Hz, 2H), 7.19 (dd, J1=2.0 Hz, J2=8.8 Hz, 2H), 6.21 (d, J=6.1 Hz, 1H), 3.65 (br-s, 4H), 2.48 (br-s, 4H), 2.34 (s, 3H) ESI/MS m/z 441.2 (M+H).

### 2-(3-(2-(4-(4-cyclohexylpiperazine-1-carbonyl)phenylamino)pyrimidin-4-ylamino) phenyl)acetonitrile (Compound 1-16)

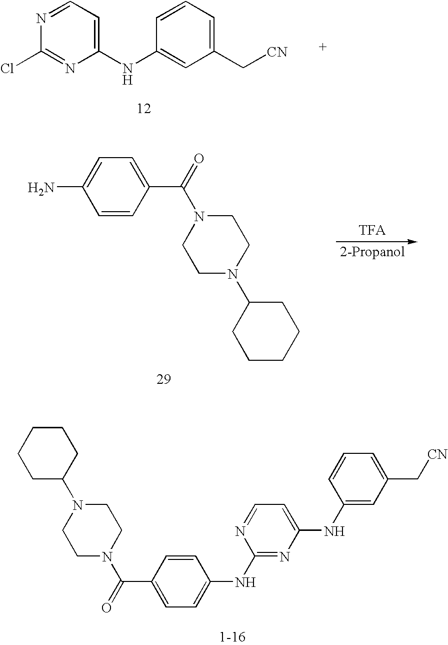

A mixture of 12 (50 mg, 0.204 mmol), (4-aminophenyl)(4-cyclohexylpiperazinomethanone (29) (58.7 mg, 0.204 mmol) in 2-propanol (5 mL) and catalytic TFA was refluxed for 24 h. NaOH was added and TLC (10% MeOH/DCM) showed some starting material and an additional new spot. Concentration and CombiFlash purification (4 g, DCM to 15% MeOH/DCM) gave Compound 1-16 (95 mg) as a brown solid.

¹NMR (400 MHz, CD₃OD) 7.99 (d, J=5.8 Hz, 1H),7.73 (m, 3H), 7.55 (d, J=8.2 Hz, 1H), 7.35 (m, 3H), 7.05 (d, J=7.9 Hz, 1H), 6.24 (d, J=6.2 Hz, 1H), 3.80 (s, 2H), 3.699br-s, 4H), 2.75 (br-s, 4H), 2.48 (br-s, 1H), 1.95 (br-s, 2H), 1.83 (br-s, 2H), 1.65 (d, J=13.0 Hz, 1H), 1.29 (br-s, 4H) ESI/MS m/z 496.1 (M+H).

### 2-(3-(2-(4-(4-methylpiperazine-1-carbonyl)phenylamino)pyrimidin-4-ylamino)phenyl)acetonitrile (Compound 1-17)

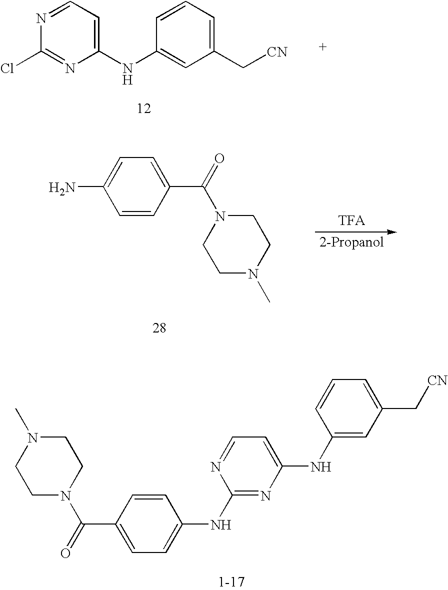

A mixture of 12 (50 mg, 0.204 mmol) and 28 (44.8 mg, 0.204 mmol) in 2-propanol (5 mL) and catalytic TFA was refluxed for 24 h. NaOH was added and TLC (10% MeOH/DCM) showed some starting material and a new compound spot. Concentration and CombiFlash purification (4 g, DCM to 15% MeOH/DCM) gave Compound 1-17 (52 mg) as a yellow solid. ¹NMR (400 MHz, DMSO-d₆) 9.53 (s, 1H), 9.40 (s, 1H), 8.06 (d, J=5.8 Hz, 1H), 7.78 (m, 3H), 7.58 (s, 1H), 7.31 (m, 3H), 6.98 (d, J=7.1 Hz, 1H), 6.27 (d, J=5.5 Hz, 1H), 4.02 (s, 2H), 3.48 (br-m, 4H), 2.42 (br-m, 4H), 2.28 (br-s, 3H) ESI/MS m/z 428.2 (M+H).

### 2-(3-(2-(4-(4-methylpiperazin-1-yl)-3-(trifluoromethyl)phenylamino)pyrimidin-4-ylamino)phenyl)acetonitrile (Compound 1-18)

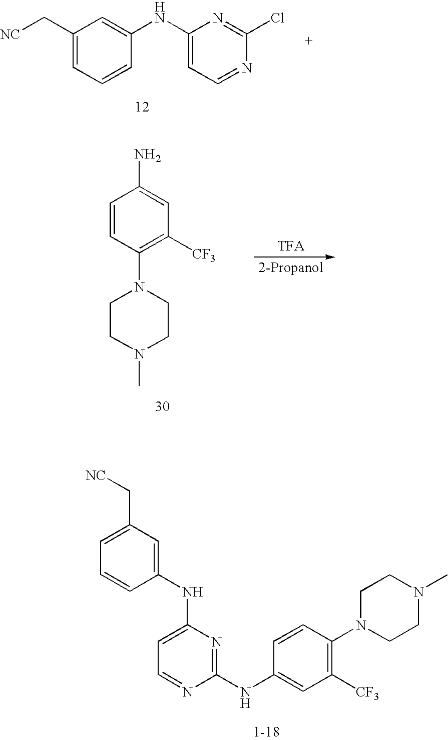

A mixture of 12 (50 mg, 0.204 mmol) and 4-(4-methylpiperazin-1-yl)-3-(trifluoromethyl)aniline (29) (46.6 mg, 0.409 mmol) in 2-propanol (5 mL) and catalytic TFA was subjected to microwave conditions at 130° C. for 2.5 h. TLC confirmed the presence of a new spot. The reaction mixture was quenched by the addition of NaOH, concentrated and purified by CombiFlash purification (4 g C-18, 5% to 100% Water/CH₃CN), and the fractions were checked by HPLC. The pure fractions were combined and concentrated to give 4.5 mg of Compound 1-18 as a pale-white solid. ¹H-NMR-(300 MHz, CDCl₃-CD₃OD) 7.62 (d, J=6.0 Hz, 1H), 7.50 (s, 1H), 7.46 (d, J=9 Hz, 1H), 7.24 (s, 2H), 7.02 (m, 2H), 6.70 (d, J=7.2 Hz, 1H), 5.89 (d, J=5.8 Hz, 1H), 3.45 (s, 1H), 3.35 (s, 1H), 3.03 (s, 2H), 2.61 (s, 4H), 2.29 (s, 4H), 2.05 (s, 3H). ESI/MS m/z 468.36 (M+H).

Salt Forms of Representative Compounds

Representative salt forms of Compounds 1-11 and 1-18 (i.e., the HCl, sulfate, mesylate and besylate forms) were prepared according to the following procedures, and listed in Table 2 below.

### 2-(3-(2-(3-fluoro-4-(4-methylpiperazin-1-yl)phenylamino)pyrimidin-4-ylamino)phenyl)acetonitrile dihydrochloride (Compound 1-11 2HCl)

Compound 1-11 (2.06 g) was suspended in 125 mL of 2-propanol. Concentrated HCl (2.47 mL, 6 eq) was added. The mixture was heated until a clear solution was obtained. The solution was cooled down at room temperature before it was moved to a −20° C. freezer. After 3 days, filtration under Argon and washing with ether afforded the bis-HCl salt of Compound 1-11 as 1.823 g of yellow powder, yield 70%.

### 2-(3-(2-(3-fluoro-4-(4-methylpiperazin-1-yl)phenylamino)pyrimidin-4-ylamino)phenyl)acetonitrile sulfate (Compound 1-11 Sulfate)

350 mg of Compound 1-11 was dissolved into 35 mL of acetone, and 0.122 mL (2.5 eq) of H₂SO₄ was added. Precipitate formed quickly. The mixture was stored at room temperature overnight. Filtration afforded 394 mg of yellow hygroscopic powder. The solid was suspended into ethanol for 1 hour and filtration afforded 350 mg of white powder, yield 68% (not hygroscopic). ¹H-NMR 300 MHz, CD₃OD) 7.94 (d, J=7.33 Hz, 1H), 7.76 (s, 1H), 7.65 (d, J=8.3 Hz, 1H), 7.49 (m, 2H), 7.29 (m, 3H), 6.57 (d, J=7.33 Hz, 1H), 3.96 (s, 2H), 3.74 (t, J=12.45 Hz, 4H), 3.42 (m, 4H), 3.12 (s, 3H), ¹⁹F-NMR (300 Mhz, CD₃OD)-186.99 Hz. Elemental analysis, Cal: C, 45.02; H, 4.60; N, 15.98; S, 10.45. Found: C, 45.68; H, 4.30; N, 15.96; S, 9.39.

### 2-(3-(2-(3-fluoro-4-(4-methylpiperazin-1-yl)phenylamino)pyrimidin-4-ylamino)phenyl)acetonitrile methanesulfonate (Compound 1-11 Mesylate)

To a warm solution of Compound 1-11 (400 mg) in 40 mL of IPA was added methylsulfonic acid (0.186 mL, 3 eq). The clear solution turned cloudy gradually and the mixture was stored at −20° C. overnight. The residue was filtered under N₂ and dried under vacuum to afford 400 mg of white powder, yield 68%.

¹H-NMR (300 MHz, CD₃OD) 7.83 (d, J=7.08 Hz, 1H), 7.67 (s, 1H), 7.55 (d, J=8.55 Hz, 1H), 7.42 (m, 2H), 7.19 (m, 3H), 6.43 (d, J=7.33 Hz, 1H), 3.85 (s, 2H), 3.62 (d, J=11.23 Hz, 4H), 3.37 (m, 4H), 3.18 (d, J=1.0 Hz, 2H), 3.04 (s, 3H), 2.71 (s, 6H). Elemental Analysis. Cal: C, 49.25; H, 5.29; N, 16.08; S, 10.52. Found: C, 48.75; H, 5.50; N, 15.13; S, 10.51.

### 2-(3-(2-(3-fluoro-4-(4-methylpiperazin-1-yl)phenylamino)pyrimidin-4-ylamino)phenyl)acetonitrile benzenesulfonate (Compound 1-11 Besylate)

To a solution of Compound 1-11 (400 mg) in 30 mL of ethanol was added benzenesulfonic acid (455 mg, 3 eq). A clear solution formed before white precipitate appeared quickly. The residue was filtered overnight under N₂ dried under vacuum to afford 520 mg of white solid, yield 74%. ¹H-NMR (300 MHz, CD₃OD-CDCl₃) 7.93 (m, 3H), 7.82 (d, J=7.08 Hz, 1H), 7.66 (s, 1H), 7.49M, 5H), 7.27 (d, J=7.33 Hz, 1H), 7.21 (d, J=9.04 Hz, 1H), 7.04 (t, J=7.3 Hz, 1H), 6.49 (d, J=7.32 Hz, 1H), 3.85 (s, 2H), 3.67 (d, J=10.2 Hz, 2H), 3.63 (d, J=11.7 Hz, 2H), 3.39 (s, 4H), 3.03 (s, 3H). Elemental Analysis, Cal: C, 57.28; H, 4.94; N, 13.36; S, 8.74. Found: C, 57.06; H, 4.86; N, 13.20; S, 8.66.

### 2-(3-(2-(4-(4-methylpiperazin-1-yl)-3-(trifluoromethyl)phenylamino)pyrimidin-4-ylamino)phenyl)acetonitrile hydrochloride (Compound 1-18HCl)

Compound 1-18 (500 mg) was difficult to completely dissolve into 40 mL of IPA. However, after adding HCl (0.446 mL, 5 eq) and heating, a clear yellow solution was obtained. The solution was cooled down to room temperature and moved to a −20° C. freezer for two days. Filtration and drying under vacuum afforded 570 mg of powder, yield 84%. ¹H-NMR (300 MHz, CD₃OD) 7.87 (d, J=7.32 Hz, 1H), 7.81 (s, 2H), 7.60 (m, 3H), 7.36 (m, 1H), 7.22 (d, J=7.82 Hz, 1H), 6.48 (d, J=7.33 Hz, 1H), 3.89 (m, 3H), 3.60 (d, J=7.57 Hz, 2H), 3.25 (m, 4H), 2.99 (s, 3H), 1.14 (d, J=6.35 Hz, 6H, IPA). Elemental Analysis, Cal: C, 53.17; H, 6.06; N, 15.50; Cl, 11.21. Found: C, 53.27; H, 6.37; N, 14.84; Cl, 10.81.

### 2-(3-(2-(4-(4-methylpiperazin-1-yl)-3-(trifluoromethyl)phenylamino)pyrimidin-4-ylamino)phenyl)acetonitrile sulfate (Compound 1-18 Sulfate)

To a solution of Compound 1-18 (400 mL) in 50 mL of IPA was added H₂SO₄ (0.137 mL, 3 eq). Precipitate formed quickly. Filtration under N₂ over the weekend afforded 407 mg of yellow powder, yield 68%. ¹H-NMR (300 MHz, CD₃OD) 7.89 (d, J=7.32 Hz, 1H), 7.78 (m, 2H), 7.60 (m, 3H), 7.36 (t, J=7.32 Hz, 1H), 7.22 (d, J=7.08 Hz, 1H), 6.48 (d, J=7.33 Hz, 1H), 3.87 (s, 2H), 3.60 (d, J=7.1 Hz, 2H), 3.27 (m, 4H), 3.21 (s, 3H). Elemental analysis, Cal: C, 44.15; H, 4.65; N, 14.13; S, 9.24. Found: C, 44.20; H, 4.65; N, 13.98; S, 9.16.

### 2-(3-(2-(4-(4-methylpiperazin-1-yl)-3-(trifluoromethyl)phenylamino)pyrimidin-4-ylamino)phenyl)acetonitrile methanesulfonate (Compound 1-18 Mesylate)

To a solution (almost clear) of Compound 1-18 (400 mg) in 20 mL of acetone was added methylsulfonic acid (0.139 mL, 2.5 eq). The solution became cloudy. The mixture was stored at −20° C. over the weekend. The acetone was poured out. The solid was washed and dried under vacuum to give 429 mg of a yellow powder, yield 74%. ¹H-NMR (300 MHz, CD₃OD) 7.88 (d, J=7.33 Hz, 1H), 7.78 (m, 2H), 7.59 (m, 3H), 7.35 (t, J=7.32 Hz, 1H), 7.22 (d, J=7.08 Hz, 1H), 6.48 (d, J=7.33 Hz, 1H), 3.86 (s, 2H), 3.60 (d, J=7.57 Hz, 2H), 3.27 (m, 4H), 2.99 (s, 3H), 2.72 (s, 6H). Elemental analysis Cal: C, 46.08; H, 5.06; N, 14.47; S, 9.46. Found: C, 46.08; H, 4.93; N, 14.16; S, 10.25.

### 2-(3-(2-(4-(4-methylpiperazin-1-yl)-3-(trifluoromethyl)phenylamino)pyrimidin-4-ylamino)phenyl)acetonitrile benzene sulfonate (Compound 1-18 Besylate)

To a hot suspension of Compound 1-18 (400 mg) in IPA (30 mL) was added benzenesulfonic acid (406 mg, 3 eq). It was cooled at room temperature and stored at −20° C. overnight. Filtration under N₂ gave 516 mg of a yellow powder, yield 72%. ¹H-NMR (300 MHz, CD₃OD) 7.81 (m, 7H), 7.58 (m, 3H), 7.43 (m, 7H), 7.19 (d, J=7.57 Hz, 1H), 6.47 (d, J=7.33 Hz, 1H), 3.84 (s, 2H), 3.58 (d, 2H), 3.23 (m, 5H), 2.97 (s, 3H), ¹⁹F-NMR (300 Mz, CD₃OD)-126.58. Elemental Analysis Cal: C, 51.60; H, 5.05; N, 11.70; S, 7.65. Found: C, 51.57; H4.96; N, 10.98; S, 7.61.

TABLE 2
Representative Salt Forms

| Compound | |
|---|---|
| No. | Structure |
| 1-11 2 HCl | 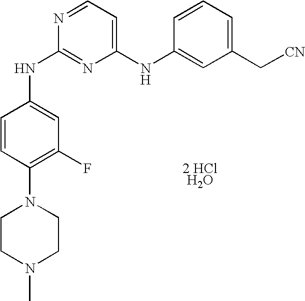 |
| | Chemical Formula: C₂₃H₃₀Cl₂FN₇O₂ |
| | Molecular Weight: 526.43 |
| 1-11 Sulfate | 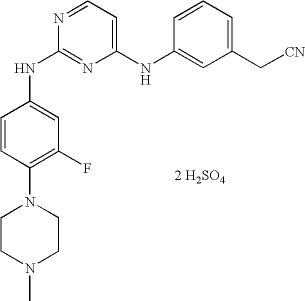 |
| | Chemical Formula: C₂₃H₂₈ FN₇O₈S₂ |
| | Molecular weight: 613.64 |
| 1-11 Mesylate | 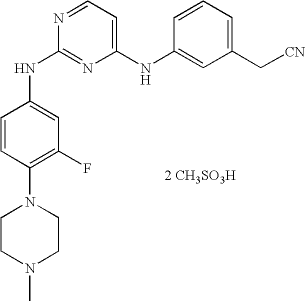 |
| | Chemical Formula: C₂₅H₃₂FN₇O₆S₂ |
| | Molecular Weight 609.69 |
| 1-11 Besylate | 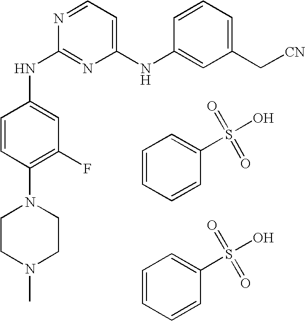 |
| | Chemical Formula: C₃₅H₃₆FN₇O₆S₂ |
| | Molecular Weight 733.83 |
| 1-18 2 HCl | 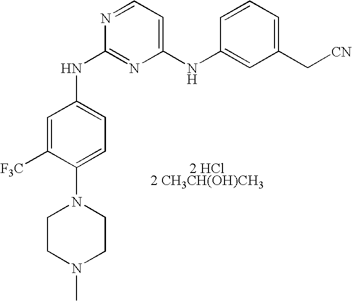 |
| | Chemical Formula: C₂₈H₃₈Cl₂F₃N₇O₂ |
| | Molecular Weight 632.55 |
| 1-18 Sulfate | 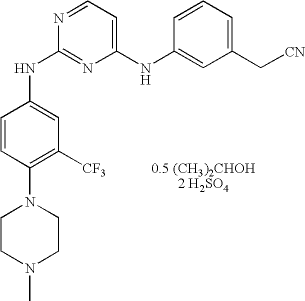 |
| | Chemical Formula: C_{25.5}H₃₂F₃N₇O_{8.5}S₂ |
| | Molecular Weight: 693.69 |
| 1-18 Mesylate | 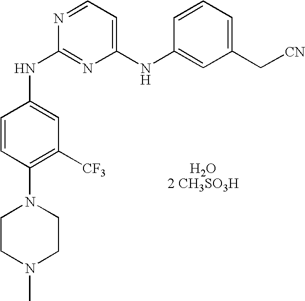 |
| | Chemical Formula: C₂₆H₃₄F₃N₇O₇S₂ |
| | Molecular Weight: 677.72 |
| 1-18 Besylate | 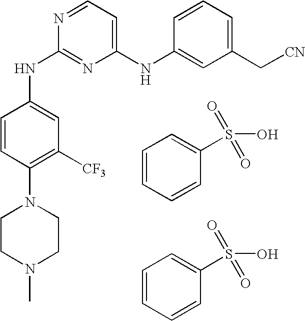 |
| | Chemical Formula: C₃₆H₄₂F₃N₇O₉S₂ |
| | Molecular Weight: 837.89 |

Activity of Representative Compounds

A. JAK2 Kinase Inhibition Assay

One illustrative manner in which JAK2 kinase activity can be determined is by quantifying the amount of ATP remaining in solution after an in vitro JAK2 kinase reaction, such as the Kinase-Glo Assay Kit (Promega, Inc., Madison, Wis.). The amount of ATP remaining in the solution after the kinase reaction serves as a substrate for the luciferase to catalyze luciferin to oxyluciferin plus one photon of light. Thus, the luminescent signal read by the Luminoskan Ascent Instrument (Thermo Electron Corp., Milford, Mass.) correlates with the amount of ATP present after the kinase reaction and inversely correlates with the amount of kinase activity. This assay is efficient at determining the IC₅₀ values of kinase inhibitors against the JAK2 kinase. These assays are set up in duplicate 50 ul volumes in white, flat bottom 96 well plates. Inhibitors are added to the solution of 1× kinase buffer, 6 uM ATP, 62.5 uM JAK2-specific substrate, 30 ng of active JAK2 enzyme, and water in serial dilutions ranging from micromolar to nanomolar concentrations. This solution is incubated at 30 degrees Celsius at 360 rpm for two hours. Following the incubation, 50 ul of Kinase-Glo reagent is added to each well, including all positive and negative control wells, and incubated at room temperature for 15 minutes. The plate is then read by the Luminoskan Ascent instrument and the results displayed with the Ascent Software version 2.6. The IC₅₀ values can then be calculated for each inhibitor tested.

Compounds may also be tested using a radiometric assay; namely, the KinaseProfile™ and IC50Profiler™ assay services (Millipore-Upstate, Dundee, UK). Briefly, compounds are tested at a single concentration (for single-point screening) or at a number of concentrations (for IC₅₀ determinations) against recombinant JAK2 enzyme in the presence of radiolabeled ATP.

The JAK2 V617F mutant isoform has recently come into focus for its role in neoplastic transformation. Thus, compounds may also be testing using the SelectScreen™ assay service (Invitrogen Corporation, Carlsbad, Calif.), which includes both wild-type and V617F mutant isoforms of JAK2. These enzymes include both the JH1 and JH2 homology regions of the protein, and differ only at amino acid 617.

B. Cell-Based JAK2 Kinase Inhibitor Assays

Cell culture-based assays can be used to evaluate the ability of compounds of the invention to inhibit one or more cellular activities, such as cancer cell growth and/or survival. Numerous cancer cell lines can be obtained from the American Type Culture Collection (ATCC) and other sources. Briefly, cells are seeded into 96-well, tissue-culture treated, opaque white plates (Thermo Electron, Vantaa, Finland), at between 600 and 14000 cells per well, depending on the speed of cell proliferation, in 100 ul of appropriate growth medium (determined by the ATCC). Cells are then exposed to the appropriate concentration of drug and allowed to grow in its presence for 96 hours. Following this, 100 ul of Cell-Titer-Glo reagent (Promega, Inc., Madison, Wis.) is added to each well. Plates are then shaken for 2 minutes at room temperature to allow for cell lysis and incubated for 10 minutes at room temperature to stabilize the luminescent signal. Similar to the Kinase-Glo assay reagent from Promega, this reagent contains both luciferase enzyme and its substrate luciferin. Luciferase, activated by ATP in the cell lysate, catalyzes the conversion of luciferin to oxyluciferin, a reaction which produces light. The amount of light produced is proportionate to the amount of ATP in the cell lysate, which is itself proportional to cell number and gives an index of cellular proliferation.

In order to detect specific inhibition of JAK2 enzyme in cell culture, Western blot assays may also be performed. For this, cells which have been treated with a potential JAK2 inhibitor are lysed with a buffer specific for the isolation and preservation of proteins (1% Nonidet P-40, 120 mM NaCl, 30 mM Tris pH 7.4, 1:100 Protease Inhibitor Cocktail III [Calbiochem/EMD Biosciences], 1:100 Phosphatase Inhibitor Cocktail 1 [Sigma-Aldrich, Saint Louis, Mo.], 1:100 Phosphatase Inhibitor Cocktail 2 [Sigma-Aldrich, Saint Louis, Mo.]). The protein concentration in these lysates is then quantified using the BCA Protein Assay Kit (Pierce). Known amounts of protein, e.g. 50 ug, are loaded onto 10% SDS-polyacrylamide gels and are subjected to reducing, denaturing SDS-PAGE. Electrophoresed proteins are transferred to a nitrocellulose membrane, which is then probed with antibodies to STAT5, pSTAT5 (Tyr 694), STAT3, and pSTAT3 (Tyr 705). As STAT5 and STAT3, at Tyrosine 694 and Tyrosine 705 respectively, are substrates for JAK2, measuring the amount of phosphorylation at these sites in treated cells provides a means by which to evaluate the efficacy of JAK2 inhibitors.

C. JAK2 Kinase Specific Activity Data

Compound Nos. 1-4, 1-7, 1-11 and 1-17 were screened at a dilution range between 300 nM and 10 nM as JAK2 inhibitors, with percent survival being determined using the Cell-Titer-Glo Assay. Each of these compounds resulted in a % survival value relative to the inhibitor concentration from which an IC₅₀ values was calculated. These compounds yielded IC₅₀ values of less than 10 nM in various cancer cell lines.

IC₅₀ values against JAK2 kinase (using the Promega Kinase-Glo assay) were measured for Compound Nos. 1-4, 1-7, 1-11 and 1-17. Each of these compounds was found to have an IC₅₀ value of less than 1 uM.

The IC₅₀ Profile™ data showed IC₅₀ values of less than 1 uM for Compound No. 1-4, 1-7, 1-11 and 1-17, while the data from compounds screened using Invitrogen SelectScreen™ profiling against wild-type (JAK2 WT) and a mutant JAK2 kinase (JAK2 V617F) also showed IC₅₀ values less than 1 μM for these four compounds.

Representative Compounds Modulate STAT3 and STAT5

A. Compound 1-4 Inhibits STAT3 and STAT5 Phosphorylation

In this example, Compound 1-4 is shown to reduce the JAK2-dependent phosphorylation of STAT3 and STAT5 in the AGS gastric cancer cell line. Briefly, AGS cells were plated in 25 cm² tissue culture flasks and incubated in the presence of varying concentrations of Compound 1-4 for 24 hours. Following incubation, cells were lysed and total protein isolated and quantified. 50 μg of total protein was electrophoresed and transferred to a nitrocellulose membrane, at which point Western Blot analysis was performed using antibodies to STAT3-phospho-Y705 and STAT5-phospho-Y694. Comparisons to total STAT3 and STAT5 were also made. Densitometry analysis was performed in order to quantify the amount of STAT3 and STAT5 phosphorylation in these treated cells. Phospho-STAT3 and phospho-STAT5 were compared to total STAT3 and STAT5 and the proportion of STAT phosphorylation relative to untreated controls was determined. Cells treated with Compound No. 1 at low micromolar concentrations (5-10 uM) exhibited reduced levels of STAT3 and STAT5 phosphorylation.

B. Compound 1-4 Reduces STAT3 Activation

When STAT3 is phosphorylated by JAK2, it forms a homodimer and is translocated to the nucleus to effect transcription of a number of target genes involved in cell proliferation. AGS gastric cancer cells were inoculated onto 96 well plates, and incubated in the presence of 5 μM Compound 1-4 for 1, 5 or 24 hours. Following incubation, cells were stained using the STAT3 HitKit (Thermo-Fisher) and detected using the Molecular Translocation BioApplication on a ArrayScan VTi high-content screening instrument (Thermo-Fisher). Nuclei were pseudostained blue (Hoechst) and STAT3 was pseudostained green (FITC).

In the untreated cells, STAT3 was found in the nucleus to a significant degree, indicating that STAT3 was phosphorylated, active, and inducing transcription. However, in cells treated with Compound 1-4, STAT3 staining was confined outside the nucleus, indicating that it was not phosphorylated and was inactive, an effect expected from an inhibitor of JAK2. Nuclear STAT3 was reduced by greater than 50% 24 h after treatment with Compound 1-4 when compared with nuclear STAT3 levels in untreated samples.

C. Compounds 1-4 and 1-7 Reduce STAT5 Phosphorylation in Cells Expressing Jak2 (V617F)

In this example, Compounds 1-4, 1-7, 1-11 and 1-17 are demonstrated to reduce the JAK2-dependent phosphorylation of STAT5 in cells expressing the V617F mutant of JAK2. Briefly, HEL cells were plated in 25 cm² tissue culture flasks and incubated in the presence of varying concentrations of Compound Nos. 1 or 2 for 24 hours. Following incubation, cells were lysed and total protein isolated and quantified. 50 μg of total protein was electrophoresed and transferred to nitrocellulose membrane, at which point Western Blot analysis was performed using an antibody to STAT5-phospho-Y694. Comparisons to total STAT5 were made. Densitometry analysis was performed in order to quantify the amount of STAT5 phosphorylation in these treated cells. From this assay, EC₅₀ values were determined to be 299 nM for Compound 1-4, 4 nM for Compound 1-7, 65.8 mM for Compound 1-11 and 266 nM for Compound 1-17.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of treatment or prevention of Castleman's disease, atherosclerosis, coronary artery disease, peripheral edema, peripheral vascular disease, glaucoma, and wet or dry age-related macular degeneration (AMD), asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration comprising:
the step of administering a therapeutically effective amount, or a prophylactically effective amount, of a compound having the following structure (I): 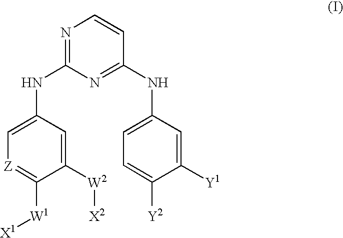
including stereoisomers and pharmaceutically acceptable salts thereof, wherein:
Z is CH or N;
W¹ and W² are independently a direct bond, —C(═O)— or —O(CH₂)ₙ—;
X¹ and X² are independently —H, —CF₃, —OCF₃, —OCHF₂, —OCH₃, —CH₃, —OH, —NO₂, —NH₂, halogen or 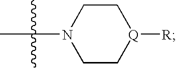
wherein Q is O or N and R is not present or R is —C₁₋₆alkyl, provided that one of X¹ or X² is 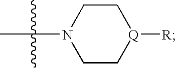
Y¹ and Y² are independently —H, —CN, halogen or a C₁₋₄alkyl group substituted with —CN, provided that Y¹ and Y² are not both —H; and
n is 1, 2 or 3.

2. The method according to claim 1, wherein in said compound Z is CH.

3. The method according to claim 2, wherein in said compound X² is halogen.

4. The method according to claim 3, wherein in said compound Y² is —CN, halogen or a C₁₋₄alkyl group substituted with —CN.

5. The method according to claim 3, wherein in said compound Y¹ is —CN, halogen, or a C₁₋₄alkyl group substituted with —CN.

6. The method according to claim 2, wherein in said compound X² is —H.

7. The method according to claim 6, wherein in said compound Y¹ is —CN, halogen, or a C₁₋₄alkyl group substituted with —CN.

8. The method according to claim 6, wherein in said compound Y² is —CN, halogen, or a C₁₋₄alkyl group substituted with —CN.

9. The method according to claim 2 wherein said compound is selected from: 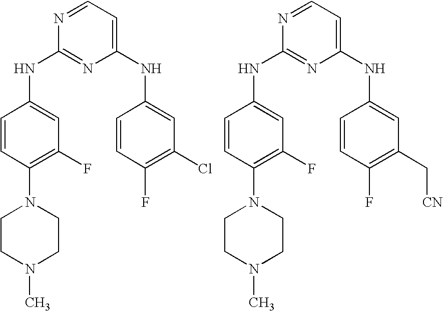
or a stereoisomer, or pharmaceutically acceptable salt thereof.

10. The method according to claim 2 wherein said compound is selected from: 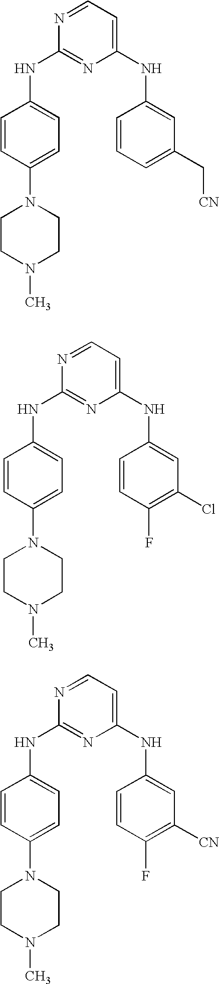
or a stereoisomer, or pharmaceutically acceptable salt thereof.

11. The method according to claim 1, wherein in said compound, or a stereoisomer, or pharmaceutically acceptable salt thereof, Z is N.

12. The method according to claim 11, wherein in said compound, or a stereoisomer, or pharmaceutically acceptable salt thereof, X² is —H.

13. The method according to claim 12, wherein in said compound, or a stereoisomer, or pharmaceutically acceptable salt thereof, Y² is —CN, halogen or a C₁₋₄alkyl group substituted with —CN.

14. The method according to claim 12, wherein in said compound, or a stereoisomer, or pharmaceutically acceptable salt thereof, Y¹ is —CN, halogen or a C₁₋₄alkyl group substituted with —CN.

15. The method according to claim 11 wherein said compound is selected from: 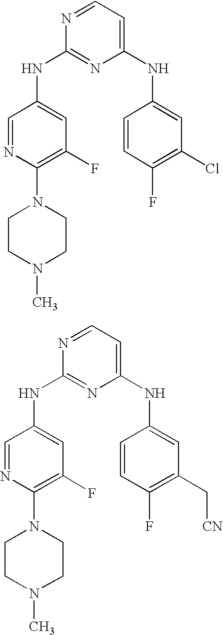
or a stereoisomer, or pharmaceutically acceptable salt thereof.

16. A method of treatment or prevention of Castleman's disease, atherosclerosis, coronary artery disease, peripheral edema, peripheral vascular disease, glaucoma, and wet or dry age-related macular degeneration (AMD), asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal, or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; alkylosing spondylitis; osteoarthritis; inflammation; or cytokine-mediated chronic tissue degeneration comprising:
the step of administering a therapeutically effective amount, or a prophylactically effective amount, of a composition comprising a compound of claim 1 in combination with a pharmaceutically acceptable excipient.

17. A method for treating a JAK2 protein kinase-mediated disease comprising administering to a subject in need thereof a therapeutically effective amount of a composition of claim 16, wherein the JAK2 protein-kinase mediated disease is asthma; chronic bronchitis; chronic obstructive pulmonary disease; adult respiratory distress syndrome; infant respiratory distress syndrome; cough; chronic obstructive pulmonary disease in animals; or adult respiratory distress syndrome.

18. The method of claim 17 wherein the JAK2 protein-kinase mediated disease is ulcerative colitis; Crohn's disease; hypersecretion of gastric acid; bacterial, fungal, or viral induced sepsis or septic shock; endotoxic shock; laminitis or colic in horses; spinal cord trauma; or head injury.

19. The method of claim 17, wherein the JAK2 protein-kinase mediated disease is neurogenic inflammation; pain; reperfusion injury of the brain; psoriatic arthritis; rheumatoid arthritis; alkylosing spondylitis; osteoarthritis; inflammation; or cytokine-mediated chronic tissue degeneration.

20. The method of claim 17, wherein the JAK2 protein-kinase mediated disease is Castleman's disease.

21. The method of claim 17, wherein the JAK2 protein-kinase mediated disease is glaucoma, dry age-related macular degeneration, or wet age-related macular degeneration.
